(19) United States
(12) Patent Application Publication
(10) Pub. No.: US 2006/0188993 A1
(43) Pub. Date: Aug. 24, 2006

(54) Method of truncating both ends of a large piece of DNA

(76) Inventor: Pradeep Kumar Chatterjee, Durham, NC (US)

Correspondence Address: CHARLES EDISON SMITH, 1512 S. ALSTON AVE., DURHAM, NC, 27707, US

(21) Appl. No.: 11/352,462

(22) Filed: Feb. 10, 2006

Related U.S. Application Data
(60) Provisional application No. 60/651,857, filed on Feb. 10, 2005.
(60) Provisional application No. 60/651,853, filed on Feb. 10, 2005.
(60) Provisional application No. 60/651,858, filed on Feb. 10, 2005.

(51) Int. Cl. C12N 15/74

(52) U.S. Cl. 435/473

(57) ABSTRACT

A method of truncating both ends of a DNA insert flanked by two different loxP sequences using transposons carrying corresponding loxP sequences pertaining to the two ends

### CROSS-REFERENCE TO RELATED APPLICATIONS

Applicant is claiming benefit of provisional application No. 60/651,857 filed Feb. 10, 2005; provisional application No. 60/651,853 filed Feb. 10, 2005 and provisional application No. 60/651,858 filed Feb. 10, 2005 under 35 U.S.C. § 119(e).

This invention was made with government support under MBRS-SCORE grant No. SO 608049 by NIGMS, grant No. 1U56CA92077-01 by NCI, and EXPORT grant #1P20 MD00175-01 by NIH. The government has certain rights in this invention.

A Sequence Listing computer program listing appendix is submitted herewith on a compact disc is hereby incorporated by reference and made a part of this application.

### BACKGROUND OF THE INVENTION

A comparison of genome sequences indicates much of the DNA that is highly conserved between human and mouse do not actually code for proteins (1), but is thought to be involved in regulating gene expression (2, 3). Part of this conserved non-coding DNA comprises Cis-acting sequences, sometimes located far away from the coding region of a gene, regulating transcription (2-5). Identifying these functionally presents a major challenge because traditional approaches have addressed only short range interactions between DNA sequence modules in small plasmids.

Localizing regulatory sequences distal to a gene functionally requires that the gene and its regulators be housed in large insert clones such as bacterial artificial chromosomes (BACs) or P1-derived artificial chromosome (PACs) (6-8). Libraries of clones with large inserts and high coverage of human, mouse and rat DNA exist (9-11), and the availability of a set of BAC clones spanning the entire human genome has set the stage for serious functional mapping projects (12). Procedures capable of truncating large genomic inserts in clones from these libraries should facilitate such effort (13 -19).

Methods that progressively delete DNA from one end of genomic inserts in BACs and PACs have been described, and used to localize genetic markers on a physical map of the chromosome (20, 21). Cre recombination of a randomly transposed loxP site in insert DNA with one endogenous to the clone generates a deletion series. The resulting truncations are recovered by packaging in a P1 phage, with the size of clones limited to 110 kb. Despite this size limitation, the ease of scanning 100 kb of conserved non-coding DNA for gene regulatory sequences quickly is a distinct advantage, and the procedure was used recently to functionally identify three new enhancer sequences 27 kb upstream of the Nkx2-5 gene in transgenic mice (3).

Because regulatory elements can sometimes be within introns or located downstream of a gene, it would be desirable to be able to truncate BAC inserts from both ends. Fortunately cloning vehicles, such as the pBACe3.6 and pTARBAC1 or -2 vectors, used for the newer resource libraries contain the insert DNA flanked by two different loxP sites (22). An arrangement such as this might have been considered ideal for deleting DNA from both ends with transposons carrying the different loxP sites had results from several subsequent studies not discouraged developing such strategies: cross-recombination between wild type and the mutant loxP511 site that flank DNA inserts in these clones, have been reported to range from 5 and 100% in a variety of settings (19, 23-26).

A recent study using a pair of different loxP site mutants suggests the earlier findings might have been influenced by excessive and/or sustained levels of Cre protein from constitutive expression, during recombination (27). Cross-recombination between wild type and 511-loxP sites was therefore reinvestigated using transient expression of Cre protein as occurs during phage P1 transduction. We now report that the leakage in recombination between these two sites was found to be no more than 0.5% of that between two wild type sites. The results enabled progressive deletion of both ends of genomic inserts in pBACe3.6 and related vectors using transposons that carry either a wild type or a mutant loxP511 site.

Large insert DNA clones propagated in bacteria or yeast have played a pivotal role in sequencing and localizing genes on a physical map of the human genome (45-48). Unfortunately, these resources could not be used directly to pursue functional studies in human cells as they lacked mammalian cell-responsive control or reporter elements. Much effort has therefore gone into either retrofitting clones from such libraries to make them amenable to analysis in mammalian cells (13,15, 16, 39, 23,18, 49,50) or alternatively, reconstruct genomic libraries in shuttle vectors that can be propagated in both bacterial and human cells (32,51,52,53,54). Progressive deletions from an end of DNA inserts in BACs and PACs using a loxP transposon have been described (14, 20), and used in mapping genetic markers on a physical map of the chromosome (20). The ability to truncate genomic DNA from both ends should greatly facilitate mapping transcription regulatory sequences that sometimes operate over large distances, define gene boundaries, and make available precisely trimmed genes in their chromosomal contexts for numerous applications. The adaptability of the deletion mapping procedure to truncate DNA from both ends using wild type and mutant loxP transposons was therefore explored.

Cre-recombination of wild type and several single base substitution mutants have shown that a loxP site recombines only with its identical copy barring a few exceptions (33). The recombination can tolerate base changes in the 8 bp spacer region, including double base substitutions, but identical pairs were required for the reaction in vitro (25). Recent studies in cells however arrived at a different conclusion: a tagged wild type Cre protein recombined a wild type loxP site containing plasmid to wild type and mutant loxP 511 sites flanking insert DNA in a BAC with equal efficiency (23). Another report analyzing several loxP mutant sites described cross-recombination between wild type and mutant ones, including the two used here, to be in the range of 1-12% (24). Although several BAC libraries with wild type and mutant loxP 511 sites flanking genomic DNA have become available (35, 10), truncation of insert DNA from both ends has not been reported. Effort was therefore directed first on resolving the dilemma over recombining wild type and mutant loxP sites with wild type Cre protein. The results were then used to develop a procedure to delete large-insert DNA from both ends in several PAC clones.

Gene expression in higher eucaryotes is often regulated at multiple stages to ensure that an appropriate level of a gene product is made at the appropriate time and only in the appropriate tissue. Many of these control mechanisms operate at the transcriptional level. Thus many mammalian genes contain tissue-specific enhancers that respond to transcription factors exclusive to the tissue to turn on expression of the gene. Such tissue-specific enhancers are often located at large distances from the coding region of the gene (57-62). Regulatory sequences silencing a gene or a chromosomal locus transcriptionally are known, and many of these also operate over large distances (63,64).

Functional analysis of distal regulatory sequences is facilitated when the gene of interest is housed in large-insert clones such as PACs and BACs. Truncations from an end of the insert DNA in such clones can be readily made using a transposon retrofitting procedure (14). The regulatory element can then be localized by analyzing expression of the gene in such truncated forms of the clone in suitable mammalian cell lines (65). LoxP-transposons capable of introducing mammalian cell-specific antibiotic-resistance genes into BAC/PAC deletions were therefore constructed for the purpose (14). Large deletions of specific sequences in BACs have also been engineered using an inducible homologous recombination system in bacteria (13, 15, 16-66, 68, 18). However these and other sequence homology based procedures (68) do not produce an entire array of deletion clones for analysis from a single experiment.

Recent advances in transgenic mouse technology allow introduction of entire BAC clones into mice (18, 58). Such procedures enable one to obtain tissue-specific expression patterns reminiscent of the endogenous gene in the animal, something not achieved earlier with introducing small plasmid constructs containing only cDNA copies of the gene (13). The technology was recently extended to locate the boundaries of regulation of the Nkx2-5 gene in vivo (69). Considerable effort has been put into identifying the regulatory elements controlling expression of the highly conserved homeobox transcription factor gene Nkx2-5 which is known to have a pivotal role in heart development (59, 60). The seven proximal regulatory elements in the Nkx2-5 gene identified in these studies using conventional technology, showed a highly complex and modular regulation pattern during development of the chambers of the mammalian heart. However, the left ventricular, atrial and tongue muscle enhancers remained unidentified in these studies.

A series of truncations in far upstream regions of the mouse Nkx 2-5 gene in a GFP BAC fusion clone was therefore used to functionally map long-range transcription regulatory sequences.

Three novel enhancer regions UH4, UH5 and UH6 located some 27 kb upstream of the gene that direct Nkx2-5 expression in a chamber/ tissue-specific manner were identified (Chi et al ms submitted). The approach necessitated that deletions created by the loxP-transposon retrofitting procedure not introduce additional promoter elements into the BAC clone so as to avoid masking those endogenous to the gene. New Tn10 mini-transposons devoid of drug resistance genes and/or eucaryotic promoter elements, pTnMarkerless1 and pTnMarkerless2 were therefore constructed for use in this study. Being devoid of bacterial antibiotic resistance genes in the transposing piece of DNA, these new transposons used a novel P1 headfal packaging mechanism to select for the otherwise low frequency of insertions.

### REFERENCES

1. The Mouse Genome Sequencing Consortium, 2002. Initial sequencing and comparative analysis of the mouse genome. Nature 420: 520-562.

2. Mortlock D P, Guenther C, Kingsley D M. (2003) A general approach for identifying distant regulatory elements applied to the Gdf6 gene. Genome Res. 9:2069-2081.

3. Chi, X., Chatteijee, P. K., Wilson III, W., Zhang, S-X., DeMayo, F., and Schwartz, R. J. (2005). Complex Cardiac Nkx2-5 Gene Expression Activated By Noggin Sensitive Enhancers Followed By Chamber Specific Modules. Proc. Natl. Acad. Sci USA (in press).

4. Jessen, J. R., Willett, C. E., and Lin, S. (1999) Artificial chromosome transgenesis reveals long-distance negative regulation of rag1 in zebrafish. Nat Genet. 23: 15-16.

5. Carvajal, J. J., Cox, D., Summerbell, D., and Rigby, P. W. (2001) A BAC transgenic analysis of the Mrf4/Myf5 locus reveals interdigitated elements that control activation and maintenance of gene expression during muscle development. Development. 128: 1857-1868.

6. Shizuya, H., Birren, B., Kim, U.-J., Mancino, V., Slepak, T., Tachiiri, Y. and Simon, M. (1992) Cloning and stable maintenance of 300-kilobase-pair fragments of human DNA in Escherichia coli using an F-factor-based vector. Proc. Natl. Acad. Sci USA 89: 8794-8797.

7. Ioannou, P. A., Amemiya, C. T., Games, J., Kroisel, P. M., Shizuya, H., Chen, C., Batzer, M. A. and de Jong, P. J. (1994) A new bacteriophage P1-derived vector for the propagation of large human DNA fragments. Nature Genet. 6: 84-89.

8. Osoegawa K., Woon P. Y., Zhao B., Frengen E., Tateno M., Catanese J. J. de Jong P. J. (1998). An improved approach for construction of bacterial artificial chromosome libraries Genomics 52:1-8.

9. Osoegawa K, Tateno M, Woon P Y, Frengen E, Mammoser A G, Catanese J J, Hayashizaki Y, de Jong P J. (2000). Bacterial artificial chromosome libraries for mouse sequencing and functional analysis. Genome Res. 10: 116-128.

10. Osoegawa K, Mammoser A G, Wu C, Frengen E, Zeng C, Catanese J J, de Jong P J. (2001). A bacterial artificial chromosome library for sequencing the complete human genome. Genome Res. 11: 483-496.

11. Osoegawa K, Zhu B, Shu C L, Ren T, Cao Q, Vessere G M, Lutz M M, Jensen-Seaman M I, Zhao S, de Jong P J (2004). BAC resources for the rat genome project. Genome Res. 4:780-785.

12. Krzywinski M, Bosdet I, Smailus D, Chiu R, Mathewson C, Wye N, Barber S, Brown-John M, Chan S, Chand S, Cloutier A, Gim N, Lee D, Masson A, Mayo M, Olson T, Pandoh P, Prabhu A L, Schoenmakers E, Tsai M, Albertson D, Lam W, Choy C O, Osoegawa K, Zhao S, de Jong P J, Schein J, Jones S, Marra M A. (2004). A set of BAC clones spanning the human genome. Nucleic Acids Res. 32: 3651-3660.

13. Yang X W, Model P, Heintz N. (1997). Homologous recombination based modification in Escherichia coli and germline transmission in transgenic mice of a bacterial artificial chromosome. Nat Biotechnol 9: 859-865.

14. Chatterjee, P. K., Coren, J. C. (1997). Isolating large nested deletions in PACs and BACs by in vivo selection of P1 headful-packaged products of Cre-catalyzed recombination between the loxP site in PAC and BAC and one introduced in transposition. Nuc. Acids Res. 25, 2205-2212.

15. Jessen J R, Meng A, McFarlane R J, Paw B H, Zon L I, Smith G R, Lin S. (1998). Modification of bacterial artificial chromosomes through chi-stimulated homologous recombination and its application in zebrafish transgenesis. Proc Natl Acad Sci USA. 95: 5121-5126.

16. Zhang Y, Buchholz F, Muyrers J P, Stewart A F. (1998). A new logic for DNA engineering using recombination in Escherichia coli. Nat Genet. 20: 123-128.

17. Muyrers J P, Zhang Y, Testa G, and Stewart A F (1999). Rapid modification of Bacterial Artificial Chromosomes by ET recombionation. Nucleic Acids Res 27: 1555-1557.

18. Gong S, Yang X W, Li C, Heintz N. (2002) Highly efficient modification of bacterial artificial chromosomes (BACs) using novel shuttle vectors containing the R6Kgamma origin of replication. Genome Res. 12: 1992-1998.

19. Nistala R, Sigmund C D. (2002) A reliable and efficient method for deleting operational sequences in PACs and BACs. Nucleic Acids Res. 30: e41.

20. Chatteijee, P. K., Yarnall, D. P., Haneline, S. A., Godlevski, M. M., Thomber, S. J., Robinson, P. S., Davies, H. E., White, N. J., Riley, J. H. and Shepherd, N. S. (1999). Direct Sequencing of Bacterial and P1 Artificial Chromosome Nested-deletions for Identifying Position-Specific Single Nucleotide Polymorphisms. Proc. Natl. Acad. Sci. (USA) 96: 13276-13281.

21. Gilmore, R. C., Baker Jr., J., Dempsey, S., Marchan, R., Corprew Jr., R. N. L., Maeda, N., Smithies, O., Byrd, G., Bukoski, R. D., Harewood, K. R. & Chatteijee, P. K. (2001). Using PAC nested-deletions to order contigs and microsatellite markers at the high repetitive sequence containing Npr3 gene locus. Gene 275: 65-72.

22. Frengen, E., Zhao, B., Howe, S., Weichenhan, D., Osoegawa, K, Gjemes, E., Jessee, J., Prydz, H, Huxley, C., deJong, P. J. (2000). Modular bacterial artificial chromosomes for transfer of large inserts into mammalian cells. Genomics 68: 118-112.

23. Wang, Z., Engler, P., Longacre, A., Storb, U. (2001). An efficient method for high-fidelity BAC/PAC retrofitting with a selectable marker for mammalian cell transfection. Genome Res. 11: 137-142.

24. Siegel, R. W., Jain, R. and Bradbury, A. (2001) Using an in vivo phagemid system to identify non-compatible loxP sequences. FEBS lett. 505: 467-473.

25. Lee, G., Saito, I., (1998). Role of nucleotide sequences of loxP spacer region in Cre-mediated recombination. Gene 216, 55-65.

26. Kolb A F. (2001) Selection-marker-free modification of the murine beta-casein gene using a lox 2272 site. Anal Biochem. 290:260-271.

27. Chatterjee, P. K., Shakes, L. A., Srivastava, D. K., Garland, D. M., Harewood, K. R., Moore, K. J., and Coren, J. S. (2004). Mutually Exclusive Recombination of Wild Type and Mutant loxP Sites in vivo Facilitates Transposon-Mediated Deletions from Both Ends of Genomic DNA in PACs. Nucleic Acids Research 32: 5668-5676.

28. Chatterjee, P. K., Mukherjee, S., Shakes, L. A., Wilson, III, W., Harewood, K. R. & Byrd, G. (2004). Selecting Transpositions of a Markerless Transposon Using Phage P1 Headful Packaging: New Transposons for Functionally Mapping Long Range Regulatory Sequences in BACs. Analytical Biochemistry 335: 305-315.

29. Chatteijee, P. K. (2004). Retrofitting BACs and PACs with LoxP Transposons to Generate Nested Deletions. “Bacterial Artificial Chromosomes” vol 1, pp 231-241. Methods in Mol. Biology series vol 255, The Humana Press Inc. Editors: Shaying Zhao & Marvin Stodolsky.

30. Stemberg N, Smoller D, Braden T. (1994). Three new developments in P1 cloning: Increased cloning efficiency, improved clone recovery, and a new P1 mouse library. Genet Anal Tech Appl. 11: 171-180.

31. Coren, J S, Pierce, J C and Stemberg N. (1995). Headful packaging revisited: the packaging of more than one DNA molecule into a bacteriophage P1 head. J. Mol. Biol. 249: 176-184.

32. Chatteijee, P. K. and Sternberg, N. L. (1996). Retrofitting high molecular weight DNA cloned in P1: introduction of reporter genes, markers selectable in mammalian cells and generation of nested deletions. Genet Anal Biomol. Eng. 13: 33-42.

33. Hoess, R. H., Wierzbicki, A., Abremski, K. (1986). The role of the loxP spacer region in Cre-mediated recombination. Nucleic Acids Res. 14: 2287-2300.

34. Chattedjee, P. K., and Briley, L. P. (2000). Analysis of a Clonal Selection Event During Transposon-mediated Nested-Deletion Formation in Rare BAC and PAC clones. Analytical Biochemistry 285: 121-126.

35. Frengen E, Weichenhan D, Zhao B, Osoegawa K, van Geel M, de Jong P J (1999). A modular, positive selection bacterial artificial chromosome vector with multiple cloning sites. Genomics 58: 250-253.

36. Stemberg N L (1992). Cloning high molecular weight DNA fragments by the bacteriophage P1 system. Trends Genet. 8: 11-16.

37. Soukharev, S, Miller J L, and Sauer, B. (1999). Segmental genomic replacement in embryonic stem cells by double lox targeting. Nucleic Acids Res. 27: e21.

38. Feng, Y Q, Seibler, J, Alami, R, Eisen, A, Westerman, K A, Leboulch, P, Fiering S, and Bouhassira, E E. (1999), Site-specific chromosomal integration in mammalian cells: Highly efficient CRE recombinase-mediated cassette exchange. J. Mol. Biol. 292: 779-785.

39. Imam A M, Patrinos G P, de Krom M, Bottardi S, Janssens R J, Katsantoni E, Wai A W, Sherratt D J, Grosveld F G. (2000). Modification of human beta-globin locus PAC clones by homologous recombination in Escherichia coli. Nucleic Acids Res. 28: E65

40. Zhang Z, Lutz B. (2002). Cre recombinase-mediated inversion using lox66 and lox71: method to introduce conditional point mutations into the CREB-binding protein. Nucleic Acids Res.30: e90.

41. Adams L D, Choi L, Xian H Q, Yang A, Sauer B, Wei L, Gottlieb D I. (2003). Double lox targeting for neural cell transgenesis. Brain Res Mol Brain Res. 110:220-233.

42. Devine S E, Boeke J D (1994). Efficient integration of artificial transposons into plasmid targets in vitro: a useful tool for DNA mapping, sequencing and genetic analysis. Nucleic Acids Res. 22: 3765-3772.

43. Abremski K, Wierzbicki A, Frommer B, Hoess R H (1986). Bacteriophage P1 Cre-loxP site-specific recombination. Site-specific DNA topoisomerase activity of the Cre recombination protein. J Biol Chem. 261: 391-396.

44. Sauer B and Henderson N. (1988) Site-specific DNA recombination in mammalian cells by the Cre recombinase of bacteriophage P1. Proc Natl Acad Sci USA 85: 5166-5170.

45. Nechiporuk T, Nechiporuk A, Sahba S, Figueroa K, Shibata H, Chen X N, Korenberg J R, de Jong P, Pulst S M. (1997). A high-resolution PAC and BAC map of the SCA2 region. Genomics. 44: 321-329.

46. Moore C S, Lee J S, Birren B, Stetten G, Baxter L L, Reeves R H (1999). Integration of cyto-genetic with recombinational and physical maps of mouse chromosome 16. Genomics. 59: 1-5.

47. Murty V V, Montgomery K, Dutta S, Bala S, Renault B, Bosl G J, Kucherlapati R, Chaganti R S. (1999). A 3-Mb high-resolution BAC/PAC contig of 12q22 encompassing the 830-kb consensus minimal deletion in male germ cell tumors. Genome Res. 9: 662-671.

48. Ahn J, Won T W, Kaplan D E, Londin E R, Kuzmic P, Gelernter J, Gruen J R. (2002). A detailed physical map of the 6p reading disability locus, including new markers and confirmation of recombination suppression. Hum Genet. 111: 339-349.

49. Kaname, T. and Huxley, C. (2001). Simple and efficient vectors for retrofitting BACs and PACs with mammalian neoR and EGFP marker genes. Gene 266: 147-153.

50. Lee E C, Yu D, Martinez de Velasco J, Tessarollo L, Swing D A, Court D L, Jenkins N A, Copeland N G. (2001). A highly efficient E. coli-based chromosome engineering system adapted for recombinogenic targeting and subdloning of BAC DNA. Genomics 73: 56-65.

51. Westphal, E. M., Sierakowska, H., Livanos, E., Kole, R., Vos, J.-M. (1998). A system for shuttling 200-kb BAC/PAC clones into human cells: stable extrachromosomal persistence and long-term ectopic gene activation. Hum Gene Ther. 9: 1863-1873.

52. Wade-Martins, R., Frampton, J., James, M. R. (1999). Long-term stability of large insert genomic DNA episomal shuttle vectors in human cells. Nucleic Acids Res. 27, 1674-1682.

53. Coren, J. S. and Sternberg, N. (2001). Construction of a PAC vector system for the propagation of genomic DNA in bacterial and mammalian cells, and subsequent generation of nested deletions in individual library members. Gene 264, 11-18.

54. Al-Hasani, K., Simpfendorfer, K., Wardan, H., Vadolas, J., Zaibak, F., Villian, R., Ioannou, P. A. (2003). Development of a novel bacterial artificial chromosome cloning system for functional studies. Plasmid 49, 184-187.

55. Gemon, A., Woldu, E., Godlevski, M., Wilson, W., Gilmore, R. C., Grant, D. J., Chatteijee, P. K., and Kephart, D. (2003). Automated Purification of Dye Terminator Sequencing Reactions: an Approach to High Throughput Capillary Electrophoresis Sequencing of Large Templates. J. Assoc. Lab. Automation 8: 19-23.

56. Hartung, M. and Kisters-Woike, B. (1998). Cre mutants with altered DNA binding properties. J. Biol. Chem. 273: 22884-22891.

57. Lee, G., and Jayaram, M. (1995). Role of partner homology in DNA recombination. Complementary base pairing orients the 5′-hydroxyl for strand joining during Flp site-specific recombination. J. Biol. Chem. 270:4042-4052.

58. Antoch, M. P., Song, E. J., Chang, A. M., Vitaterna, M. H., Zhao, Y., Wilsbacher, L. D., Sangoram, A. M., King, D. P., Pinto, L. H., & Takahashi, J. S. (1997) Functional identification of the mouse circadian Clock gene by transgenic BAC rescue. Cell 89: 655-667.

59. Yang, X. W., Wynder, C., Doughty, M. L., and Heintz, N. (1999) BAC-mediated gene-dosage analysis reveals a role for Ziprol (Ru49/Zfp38) in progenitor cell proliferation in cerebellum and skin. Nat Gene. 22: 327-335.

60. Reecy J M, Li X, Yamada M, DeMayo F J, Newman C S, Harvey R P, Schwartz R J. (1999) Identification of upstream regulatory regions in the heart-expressed homeobox gene Nkx2-5. Development 126: 839-849.

61. Tanaka M, Wechsler S B, Lee I W, Yamasaki N, Lawitts J A, Izumo S. (1999) Complex modular cis-acting elements regulate expression of the cardiac specifying homeobox gene Csx/Nkx2.5. Development. 126: 1439-1450.

62. Carvajal, J. J., Cox, D., Summerbell, D., and Rigby, P. W. (2001) A BAC transgenic analysis of the Mrf4/Myf5 locus reveals interdigitated elements that control activation and maintenance of gene expression during muscle development. Development. 128: 1857-1868.

63. Gottgens B, Barton L M, Chapman M A, Sinclair A M, Knudsen B, Grafham D, Gilbert J G, Rogers J, Bentley D R, Green A R. (2002). Transcriptional regulation of the stem cell leukemia gene (SCL)—comparative analysis of five vertebrate SCL loci. Genome Res. 5: 749-759.

64. Jessen, J. R., Willett, C. E., and Lin, S. (1999) Artificial chromosome transgenesis reveals long-distance negative regulation of rag1 in zebrafish. Nat Genet. 23: 15-16.

65. DiSepio, D., Bickenbach, J. R., Longley, M. A., Bundman, D. S., Rothnagel, J. A., and Roop, D. R. (1999) Characterization of loricrin regulation in vitro and in transgenic mice. Differentiation. 64: 225-235.

66. Brake, R. L., Chatteijee, P. K., Kees, U. R & Watt, P. M. (2004). The functional mapping of long-range transcription control elements of the HOX11 proto-oncogene. Biochem. Biophys. Res. Com. 313: 327-335.

67. Orford M, Nefedov M, Vadolas J, Zaibak F, Williamson R, Ioannou P A. (2000) Engineering EGFP reporter constructs into a 200 kb human beta-globin BAC clone using GET Recombination. Nucleic Acids Res 18: E84

68. Lee E C, Yu D, Martinez de Velasco J, Tessarollo L, Swing D A, Court D L, Jenkins N A, Copeland N G. (2001) A highly efficient Escherichia coli-based chromosome engineering system adapted for recombinogenic targeting and subcloning of BAC DNA. Genomics. 73: 56-65.

69. Swaminathan S, Ellis H M, Waters L S, Yu D, Lee E C, Court D L, Sharan S K. (2001). Rapid engineering of bacterial artificial chromosomes using oligonucleotides. Genesis. 1: 14-21.

70. Chi X, Zhang S-X, DeMayo, F. J. Rosenberg, S M and Schwartz, R. J. (2003) Expression of Nkx2-5-GFP bacterial Artificial Chromosome Transgenic Mice Closely Resembled Endogenous Nkx2-5 Gene Activity. Genesis 35: 220-226.

71. Sambrook, J., Fritsch, E. F. and Maniatis, T. (1989) Molecular Cloning: A Laboratory Manual, Cold Spring Harbor Laboratory Press, Cold Spring Harbor, N.Y. 2^{nd} edn pp1.25-1.28.

72. Elliott T, Roth J R (1988). Characterization of Tn10d-Cam: a transposition defective Tn10 specifying chloramphenicol resistance. Mol Gen Genet 213: 332-338.

73. Craig N. L. (1997). Target site selection in transposition. Annu Rev Biochem 66: 437-474.

74. Windle B E, Hays J B. (1986). A phage P1 function that stimulates homologous recombination of the Escherichia coli chromosome. Proc Natl Acad Sci USA 83:3885-3889.

75. Laufer C S, Hays J B, Windle B E, Schaefer T S, Lee E H, Hays S L, McClure M R. (1989) Enhancement of Escherichia coli plasmid and chromosomal recombination by the Ref function of bacteriophage P1. Genetics 123: 465-476.

76. Yarmolinsky, M. B. & Sternberg N. (1988). Bacteriophage P1 in The Bacteriophages, Vol 1 edited by Richard Calendar, Plenum Publishing Corporation.

77. Coren, J. S., Sternberg, N., 2001. Construction of a PAC vector system for the propagation of genomic DNA in bacterial and mammalian cells, and subsequent generation of nested deletions in individual library members. Gene 264, 11-18.

78. Pierce J C, Sternberg N, Sauer B. (1992). A mouse genomic library in the bacteriophage P1 cloning system: organization and characterization. Mamm Genome. 3: 550-558.

79. Shepherd N S, Pfrogner B D, Coulby J N, Ackerman S L, Vaidyanathan G, Sauer R H, Balkenhol T C, Sternberg N. (1994). Preparation and screening of an arrayed human genomic library generated with the P1 cloning system. Proc Natl Acad Sci USA. 91: 2629-2633.

80. Lewin B. Genes VII (2000) In Transposons, Chapter 15, Oxford Univ Press, Inc. New York.

### BRIEF SUMMARY OF THE INVENTION

Contrary to several earlier reports, it is found that cross-recombination between wild type and the mutant loxP511 sites is less than 0.5% of that between two wild type sites if Cre protein is expressed by phage P1 during an infection. The finding enables development of a procedure to truncate DNA progressively from both ends of large genomic inserts flanked by these two loxP sites in pBACe3.6 and related vectors with transposons carrying either a wild type or a loxP511 sequence. Newly constructed loxP511 transposons contained either a kanamycin resistance gene or no marker. Insert DNA ends in deletions were sequenced with primers unique to each transposon-end remaining after the respective recombination. End-sequencing 223 deletions confirmed that the low level of cross-recombination, observed between those sites during the P1 transductions, does not complicate the procedure: truncations from the unintended end of genomic inserts did not occur. Multiple BACs pooled together could also be processed in a single tube to make end-deletions. This deletion technology, utilizing the very minimal cross-recombination between the mutant and wild type loxP sites of most BAC clones in the public domain and a heterologous one inserted as a transposon, should facilitate functionally mapping long-range gene regulatory sequences and help isolate genes with defined functional boundaries in numerous projects including those of therapeutic interest.

Recombination of wild type and mutant loxP sites mediated by wild type Cre protein was analyzed in vivo using a sensitive phage P1 transduction assay. Contrary to some earlier reports, recombination between loxP sites was found to be highly specific: a loxP site recombined in vivo only with another of identical sequence, with no crossover recombination either between a wild type and mutant site; or between two different mutant sites tested. Mutant loxP sites of identical sequence recombined as efficiently as wild type. The highly specific and efficient recombination of mutant loxP sites in vivo helped develop a procedure to progressively truncate DNA from either end of large genomic inserts in PACs using transposons that carry either a wild type or mutant loxP sequence. PAC libraries of human DNA were constructed with inserts flanked by a wild type and one of two mutant loxP sites, and deletions from both ends generated in clones using newly constructed wild type and mutant loxP transposons. Analysis of the results provides new insight into the very large co-integrates formed during P1 transduction of plasmids with loxP sites: a model with tri- and possibly multimeric co-integrates comprising the PAC plasmid, phage DNA, and transposon plasmid(s) as intermediates in the cell appears best to fit the data. The ability to truncate a large piece of DNA from both ends is likely to facilitate functionally mapping gene boundaries more efficiently, and make available precisely trimmed genes in their chromosomal contexts for therapeutic applications.

New Tn10 minitransposons were constructed to functionally map long-range transcription regulatory sequences in BACs and PACs. Each contained a wild type loxP site, but significantly, no mammalian or bacterial genes and/or promoter elements within the transposed portion of DNA. In contrast to loxP transposons described earlier, the new ones do not introduce transcription regulatory elements capable of interfering with those endogenous to the BAC clone in functional mapping studies. Progressive deletions from the loxP-end of genomic DNA were efficiently generated using these transposons, and a series of truncations generated in a GFP-BAC fusion clone unambiguously identified three new long-range enhancer sequences functionally in the Nkx2-5 gene in transgenic mice. Insertions of these new transposons lacking antibiotic resistance genes into a BAC or PAC were indirectly selected by their ability to delete enough DNA from the clone so as to enable its packaging within a P1 phage head with both loxP sites intact for subsequent recovery of the large plasmid. The outcome of such an indirect mode of selection is two fold, one desirable while the other undesirable: 1) because the screen is not antibiotic resistance marker dependent, the same transposon can be used to generate nested deletions efficiently in both BACs and PACs, and 2) deletions through intra-insert recombinations unrelated to LoxP-Cre also get packaged and recovered, and size analyses of the BAC/PAC vector band after NotI digestion is indispensable to identify authentic loxP-Cre deletions. The procedure nevertheless offers a potential approach to map recombinogenic sequences in BACs and PACs.

### BRIEF DESCRIPTION OF THE DRAWINGS

FIG. 1 is a schematic diagram of new transposon plasmids containing wild-type and loxP511 sites.

FIG. 2 is a graphic display of FIGE analysis of NotI digested DNA isolated from progressive truncations.

FIG. 3 is a schematic representation of deletions made sequentially from both ends of genomic DNA insert

FIG. 4 is a graphic display of FIGE analysis of NotI digested DNA isolated from deletions made from the wild-type loxP end.

FIG. 5 is a schematic diagram showing sequencing primers usuable after first and second deletions.

FIG. 6A is a schematic diagram of P1 transduction of Tn plasmids.

FIG. 6B is a schematic diagram of new transposon plasmids containing wild-type or mutant loxP sites.

FIG. 7 is a graphic display of progressive deletions from the mutant loxP end of genomic DNA insert generated with pTnloxP*

FIG. 8A is a schematic diagram of deletions and inversions formed with pTnloxP*-1 and -2 in JCPAC-13 and -9.

FIG. 8B is a schematic diagrams of sequential deletions generated first with pTnloxPwt and then with either pTnloxP*-1 or -2 in JCPAC-13 and -9 repectively.

FIG. 9A is a graphic display of deletions from the wild-type loxP end generated with pTnloxPwt in JCPAC-9 and -13

FIG. 9B is a graphic display of deletions from the mutant loxP site end of deletion clones 1 and 77 generated with pTnloxP*-2 or -1 respectively.

FIG. 10 is a schematic diagram showing possible multi-plasmid co-integrates as intermediat3es during transduction of mutant loxP site-containing plasmids by phage P1.

FIG. 11A is a schematic diagram of eukaryotic promoterless transposons.

FIG. 11B is a schematic diagram explaining how the markerless transposon strategy works.

FIG. 11C is a schematic diagram showing the loxP transposon-induced deletion in Nkx2-5 GFP-BAC with pTnMarkerless1.

FIG. 12 is a graphic display of FIGE analysis of Nkx2-5 GFP-BAC deletions generated with pTnMarkerless1.

FIG. 13A is a graphic display of FIGE analysis of Npr3PAC deletions generated with pTnMarkerless1.

FIG. 13B is a schematic diagram of the starting Npr3PAC clone and its deletion obtained using pTnMarkerless1.

FIG. 14 is a graphic display of FIGE analysis of NotI-digested DNA from Nkx2-5 GFP-BAC-nested deletions.

FIG. 15 is a graphic display of FIGE analysis of NotI-digested DNA isolated from deletion clones generated with pTnmarkerless2 on JCPAC 9 (lanes 3-15) and JCPAC 13 (lanes 20-30).

### DETAILED DESCRIPTION OF THE PREFERRED EMBODIMENTS OF THE INVENTION

In particular, the invention herein is a method of truncating both ends of a DNA insert flanked by a wild type loxP site and a loxP511 site in a vector using loxP transposon plasmids comprising: constructing a plurality of plasmids of identical sequence each containing a first transposon carrying a loxP511 site; transforming said plurality of first transposon containing plasmids into cells, each containing a DNA insert in said vector carrying a first antibiotic resistance gene; inducing each of said plurality of first transposons to insert into said DNA inserts in said cells; infecting said cells containing said plurality of first transposons inserted into said DNA inserts with Phage P1, and wherein Cre protein is expressed by Phage P1 during said infection; truncating first end portions of each of said DNA inserts, wherein each of said DNA inserts contains one of said inserted first transposons, and thereby creating a plurality of first end truncated DNA inserts with new first ends; packaging said first end truncated DNA inserts into a P1 Phage head; using said P1 Phage head to infect a first colony of fresh NS3516 cells to amplify said first end truncated DNA inserts by selecting on LB agar plates containing a first antibiotic whose resistance gene occurs in said vector, and thereby amplifying said plurality of first end truncated DNA inserts in a plurality of NS3516 cell colonies generated from said first colony of fresh NS3516 cells; isolating said amplified plurality of different first end truncated DNA inserts in said vector to obtain a plurality of colonies, each colony with the same end truncation, from said first colony of fresh NS3516 cells; using a first synthetic DNA primer to sequence said amplified plurality of new first end portions of said amplified plurality of different first end truncated DNA inserts; constructing a plurality of plasmids of identical sequence each containing a second transposon carrying a wild type loxP site and a second antibiotic resistance gene different from said first antibiotic resistance gene occurring in said vector; transforming said plurality of second transposon containing plasmids into cells containing said amplified plurality of first end truncated DNA inserts; inducing said plurality of second transposons to insert into said amplified plurality of first end truncated DNA inserts; infecting said cells containing said plurality of second transposons inserted into said plurality of amplified first end truncated DNA inserts with Phage P1, and wherein Cre protein is expressed by Phage P1 during said infection; truncating an opposite end portion of each of said plurality of amplified first end truncated DNA inserts, wherein each of said amplified first end truncated DNA inserts contains one of said inserted second transposons and creating a plurality of doubly end truncated DNA inserts with a new opposite end; packaging said plurality of doubly end truncated DNA inserts in said vector into said P1 Phage head; using said P1 Phage head to infect a second colony of fresh NS3516 cells to amplify said plurality of doubly end truncated DNA inserts by selecting on LB agar plates containing said first antibiotic and a second antibiotic whose resistance gene exists in said second transposon, and thereby amplifying a plurality of doubly end truncated DNA inserts in a plurality of NS3516 cell colonies generated from said second colony of fresh NS3516 cells; isolating said amplified plurality of different doubly end truncated DNA inserts in said vector to obtain a plurality of colonies, each colony with the same opposite end truncation from said second colony of fresh NS3516 cells; and using a second synthetic DNA primer to sequence said amplified plurality of new opposite end portions of said different doubly end truncated DNA inserts. The plurality of plasmids may alternatively contain a transposon carrying a wild type loxP site.

The first transposon is preferably transposon plasmid pTnloxP511 (B) markerless 1, pTnloxP511 (B) markerless 2, or pTnloxP511 (A) markerless 2, and the second transposon is preferably pTn(RSVneo 2)/loxP or pTn(RSVneo 1)/loxP. The first antibiotic is preferably chloramphenicol and the preferred second antibiotic is kanamycin. The preferred vector is a BAC clone selected from the group consisting of pBACe3.6, pTARBAC 1 & 2, and pTARBAC 2.1. or from the group consisting of RP11-219A15, RPI1 -158M20, RP11-434D2, RP23-209022, RP23-92L23, RP23-366M16, RP23-10N20, RP23-124B2, RP23-444K15, and RP23-130D16.

The invention herein further comprises A method of selecting transpositions using phage P1 headful packaging instead of using an antibiotic resistance gene within a transposon comprising: constructing a plurality of plasmids of identical sequence each containing a first transposon carrying either a wild type loxP or a mutant loxP site; transforming said plurality of first transposon containing plasmids into cells, each containing a DNA insert in said vector carrying a first antibiotic resistance gene; inducing each of said plurality of first transposons to insert into said DNA inserts in said cells; infecting said cells containing said plurality of first transposons inserted into said DNA inserts with Phage P1, and wherein Cre protein is expressed by Phage P1 during said infection; truncating first end portions of each of said DNA inserts, wherein each of said DNA inserts contains one of said inserted first transposons, and thereby creating a plurality of first end truncated DNA inserts with new first ends; packaging said first end truncated DNA inserts into a P1 Phage head; using said P1 Phage head to infect a first colony of fresh NS3516 cells to amplify said first end truncated DNA inserts by selecting on LB agar plates containing a first antibiotic whose resistance gene occurs in said vector, and thereby amplifying said plurality of first end truncated DNA inserts in a plurality of NS3516 cell colonies generated from said first colony of fresh NS3516 cells; isolating said amplified plurality of different first end truncated DNA inserts in said vector to obtain a plurality of colonies, each colony with the same end truncation, from said first colony of fresh NS3516 cells; and using a first synthetic DNA primer to sequence said amplified plurality of new first end portions of said amplified plurality of different first end truncated DNA inserts.

Minimal Cross-Recombination between Wild Type and loxP511 Sites in Vivo Facilitates Truncating Both Ends of Large DNA Inserts in pBACe3.6 and Related Vectors.

Materials and Methods

The oligodeoxyribonucleotides d (GGCCGCTAATAACTT CGTATAGTATACA TTATA-CGAAGTTATTGTTTAAACC) (SEQ ID NO: 9) and d (GGCCGGTTTAAACAATAACTT CGTATAA- TGTATACTATACGAAGTTATTAGC) (SEQ ID NO:19), were annealed by heating equimolar quantities in 10 mM trisHCl pH 8.0 buffer containing 1 mM EDTA and 10 mM NaCl to 70° C. and slow cooling to room temperature. The duplex oligo contains the mutant loxP511 sequence along with a Pme-I site, and was ligated to plasmid DNA linearized with NotI enzyme.

Construction of Tn10 Minitransposons with Mutant loxP511 Sites:

The transposon plasmid pTnloxP*-1 (27), was the starting point for all mutant loxP511 site-containing plasmids. The gene for chloramphenicol resistance located near one of the seventy bp inverted repeat ends was removed by digesting the pTnloxP*-1 DNA to completion with Pvu II, gel purifying the largest fragment, and performing a partial digest with Sca I. The largest fragment was again gel purified, and the two blunt ends created by the Pvu II and Sca I enzymes ligated with high concentration T4 DNA ligase. Transformed colonies were selected on LB agar plates with ampicillin. The gene for ampicillin resistance is located outside the seventy bp inverted repeat ends of the transposon.

Next the sequences for tetracycline resistance gene and the mutant loxP*-1 site were removed by NotI digestion, and into it was ligated a duplex oligonucleotide with NotI compatible ends and containing the mutant loxP511 sequence and a Pme I restriction site (shown above). Plasmids acquiring the loxP511 oligonucleotide were screened for by digesting clone DNA with Pme I enzyme. Both orientations of the loxP511 site-inserted transposon were isolated. They were named pTnLoxP511 (A)markerless 1 and pTnLoxP511 (B)markerless 1. They are designated markerless because they do not contain any antibiotic resistance markers within the seventy bp inverted repeat ends of the transposon to select for transpositions of the loxP511 site into target DNA (see reference 28 for discussion).

An additional oligonucleotide duplex for anchoring a sequencing primer (Seq 25) was inserted into the unique Asc I site of pTnLoxP5 111 (B)markerless 1 to generate pTnLoxP5 111 (B)markerless 2. The plasmid was linearized with Asc I and the duplex obtained by hybridizing the following oligonucleotides M13C and M13D was ligated as described above.

M13C: d

(SEQ ID NO:10)

(CGCGCCTAGTAAAACGACGGCCAGTAGTCGTG ACTGGGAAAACCT
GTT-TAAACC);
M13D: d

(SEQ ID NO:11)

(CGCGGGTTTAAACAGGTTTTCCCAGTCACGACTACTGGCC-GTCGTTTT
ACTAGG).

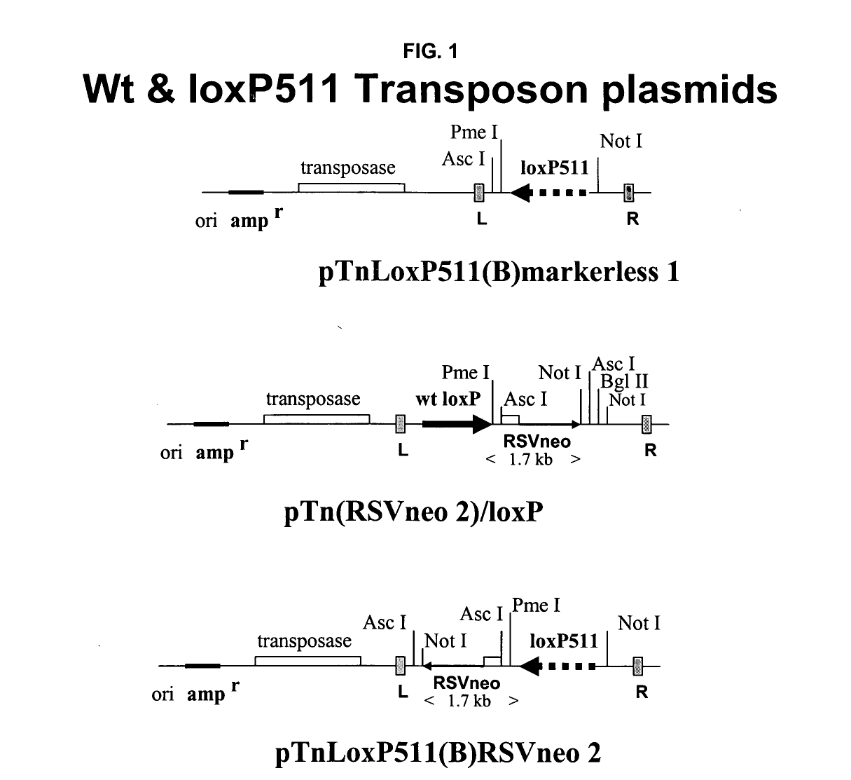

A RSV neomycin resistance gene cassette, coding for kanamycin resistance in bacteria, was excised from pTnBAC/loxP (14) with Asc I, and inserted into the AscI site of pTnLoxP511 (B)markerless 1 (FIG. 1). The resulting transposon plasmids are named pTnLoxP511 (B)RSVneo 1 or pTnLoxP511 (B)RSVneo 2 to distinguish the two orientations of the RSVneo cassette. Only pTnLoxP511 (B)RSVneo 2 is discussed here.

Construction of a Tn10 Minitransposon with Wild Type loxP Sites

The kanamycin resistance gene-containing wt loxP transposon plasmid, pTn(RSVneo 2)/loxP, was constructed by introducing a RSV neomycin gene cassette into pTnMarkerless2 described earlier (28). It differs from pTnBAC/loxP (14) in that all sequences related to the chloramphenicol resistance gene were removed from the latter, in order to reduce recombinations between the chloramphenicol resistance gene in BACs and those sequences in the pTnBAC/loxP plasmid (14) (see ref (28) for discussion). The RSV neomycin gene cassette was excised from pTnBAC/loxP with Asc I, the DNA fragment purified from an agarose gel, and ligated to pTnMarkerless2 linearized also with Asc I. Plasmids containing both orientations of the RSV neo gene cassette were isolated, pTn(RSVneo 1)/loxP and pTn(RSVneo 2)/loxP, and only the latter orientation used here and shown in FIG. 1.

Generating Deletions from the loxP511 end of Inserts in pBACe3.6 Vector-Derived Clones

The following 10 clones from the human and mouse BAC libraries were obtained from BACPAC resources, CHORI (Oakland, Calif.): RP11-219A15, RP11-158M20, RP11-434D2, RP23-209022, RP23-92L23, RP23-366M16, RP23-10N20, RP23-124B2, RP23-444K15, RP23-130D16. The genomic inserts in these clones are flanked by a wild type and a mutant loxP511 site in the BAC vector pBACe3.6 (9-11, 22).

Nested deletions using pTnLoxP511 (B)markerless 1, pTnLoxP511 (B)markerless 2 and pTnLoxP511 (B)RSVneo 2 from the loxP511 -end were generated in each of the above clones as described earlier (28, 29).

Processing Multiple BAC Clones to make End-Deletions

BACs were also pooled together and processed together in a single tube to make end-deletions. Each BAC clone was transformed separately with the transposon plasmid, and the transformed colonies grown to saturation before pooling.

End-Sequencing of BAC Deletion Clones

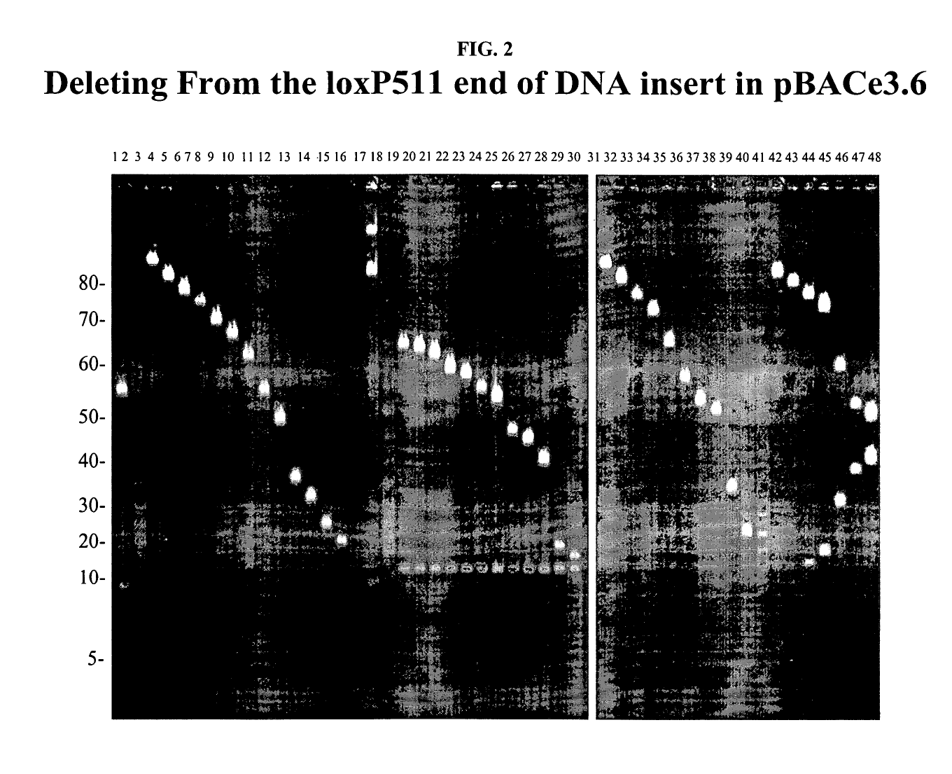

Typically, miniprep DNA was isolated from 60 clones picked randomly from the several hundred member BAC deletion library. Deletion clones were passed through an ampicillin sensitivity screen (described in ref 20) if the transposon plasmid was recovered in more than 20% of the deletions. Approximately half the clones from a first round deletion series analyzed on FIGE were of unique size. Deletions arising from intra-insert recombinations independent of loxP-Cre, seen only when using markerless transposons of either type, were weeded out after FIGE analysis (lane 1 of FIG. 2 shows an example). These are identified by their BAC vector DNA fragment identical in size to starting BACs (28). DNA from 20 clones of each deletion series was sequenced directly using a transposon-end primer (20) and Big Dye Terminator chemistry on an ABI-3100 AVANT genetic analyzer. Primer extended products were purified using Magnesil (Promega Corporation) according to procedures supplied by the manufacturer and described in (27, 28). Primers for sequencing newly created ends of deletions from the loxP511 side of insert DNA, after trimming the wild type end, have been developed. These are listed below:

Sequencing Primers

| | |
|---|---|
| Seq 8: d | |
| (GCAGTGTGACCGTGTGCTTCTCAAATGC) | (SEQ ID NO:12) |
| Seq 21: d | |
| (GATCGGCGCGCCATGATC) | (SEQ ID NO:13) |
| Seq 25: d | |
| (GCCAGTAGTCGTGACTG) | (SEQ ID NO:14) |
| Neo 8: d | |
| (GTTTTCCCTTGTCCAGATAGC) | (SEQ ID NO:15) |
| Neo 11: d | |
| (CTGAGTGCTTGCGGCAGCG) | (SEQ ID NO:16) |
| Neo 12: d | |
| (GAATCGTTTTCCGGGACG) | (SEQ ID NO:17) |
| Neo 16: d | |
| (GATCTCATGCTGGAGTTCTTCGCC) | (SEQ ID NO:18) |

Results

Truncating DNA inserts from both ends using loxP-transposons critically depends on the degree of leakage in recombination between the two loxP sites flanking the insert. Because varying degrees of recombination cross-reactivity between these sites was observed in previous studies (19, 23-26), it was important to reinvestigate this under the P1 transduction conditions actually used in our deletion procedure (27).

Estimating Recombination Cross-Reactivity between Wild Type and Mutant loxP511 Sites using Phage P1 Transduction

Several mutant loxP sites have been tested both for their recombination efficiency and exclusivity under different Cre protein-exposure conditions (23-27). Phage P1 expresses Cre early during an infection and efficiently transduces a wild type loxP plasmid with high fidelity (30-32). The ability of the wild type loxP site in P1 phage to transduce the mutant loxP511 plasmid was used as an indicator of recombination cross-reactivity between these sites (described in ref 27). Results in Table I indicate that although P1 was able to efficiently transduce the plasmid with a wild type site, it could recover the mutant loxP511 plasmid only very inefficiently (compare #17 with #21). Unlike the results with two other mutant loxP sites described earlier (27), inhibition of transduction of the loxP511 mutant was incomplete. Comparing parallel transductions we find that the loxP511 plasmid is transduced 0.1-0.5% as efficiently as the wild type. This level of recombination cross-reactivity is significantly less than that reported earlier in several in vivo and in vitro analyses (19, 23-26). The exclusivity between the two sites appears high, although not absolute as reported originally (33). Consistent with previous findings (27), phage P1 was able to efficiently transduce the loxP511 plasmid if another carrying both a wild type and the loxP511 site exists in the cell (rows 5, 6, Table I). Phage P1 transduced the loxP511 plasmid in the presence of BAC deletions, #39 and #45, generated from BAC RP23-444K15 by truncating from the wild type loxP-end with a markerless wt-loxP transposon (28).

Generating Deletions from the loxP511 end of Insert DNA in pBACe3.6

Because the 511 and wild type loxP sequences cross-recombine only slightly, insert DNA flanked by these can be deleted from both ends using transposons carrying those sites. Verifying authenticity of the newly created ends after each round of deletion should compensate for the minor leakiness. Transposons were constructed with the two types of loxP sites such that a different end of the Tn10 was left behind after deletion formation at each end of insert DNA. Unique primers were designed into each transposon-end remaining after the recombination.

Two classes of transposons were constructed carrying the loxP511 site: i) markerless and ii) with kanamycin resistance gene as marker. Only transposons of the “B” series of both classes, with the loxP511 oriented as shown in FIG. 1, were used in this study. The “A” series of both classes had the loxP511 site in the opposite orientation. As noted earlier (28), transposition of a markerless loxP-transposon is selected through P1-headful packaging and not by the transfer of an antibiotic resistance gene into the target DNA. This necessitates the starting BAC to be larger than P1 headful size. Depending on which end of the genomic insert is deleted first, the markerless transposon could carry a wild type or a mutant loxP511 site. Markerless transposons with wild type loxP sites have been described earlier (28).

The transposon plasmid pTnLoxP511(B)markerless 1 was transformed into each of the 10 BACs listed in Methods. Transformed colonies were selected on LB agar plates containing chloramphenicol plus ampicillin. Colonies from a plate were pooled and processed for making end-deletions as described earlier for wild type loxP transposon-transformed BACs (34, 28). BAC deletions were selected on chloramphenicol only plates. Miniprep DNA isolated from deletion clones were digested with NotI enzyme before analysis by FIGE (29). Lanes 3-15 of FIG. 2 show DNA isolated from clones deleted from the loxP511 end of insert in BAC RP23-444K15. Lane 1 displays an intra-insert deletion independent of loxP-Cre (28), with the vector DNA fragment size identical (8.8 kb) to starting BAC (lane 17).

Deletions from the loxP511 end were also made with pTnLoxP511(B)RSVneo 2 (FIG. 1) in a subset of the BAC clones. Lanes 19-30 FIG. 2, show NotI digested DNA from a set of these deletions with the BAC vector fragment now at 10.5 kb.

Specificity of loxP-Cre Recombinations during Deletion Formation: using size of pBACe3.6 Vector DNA Fragment with NotI as a Diagnostic

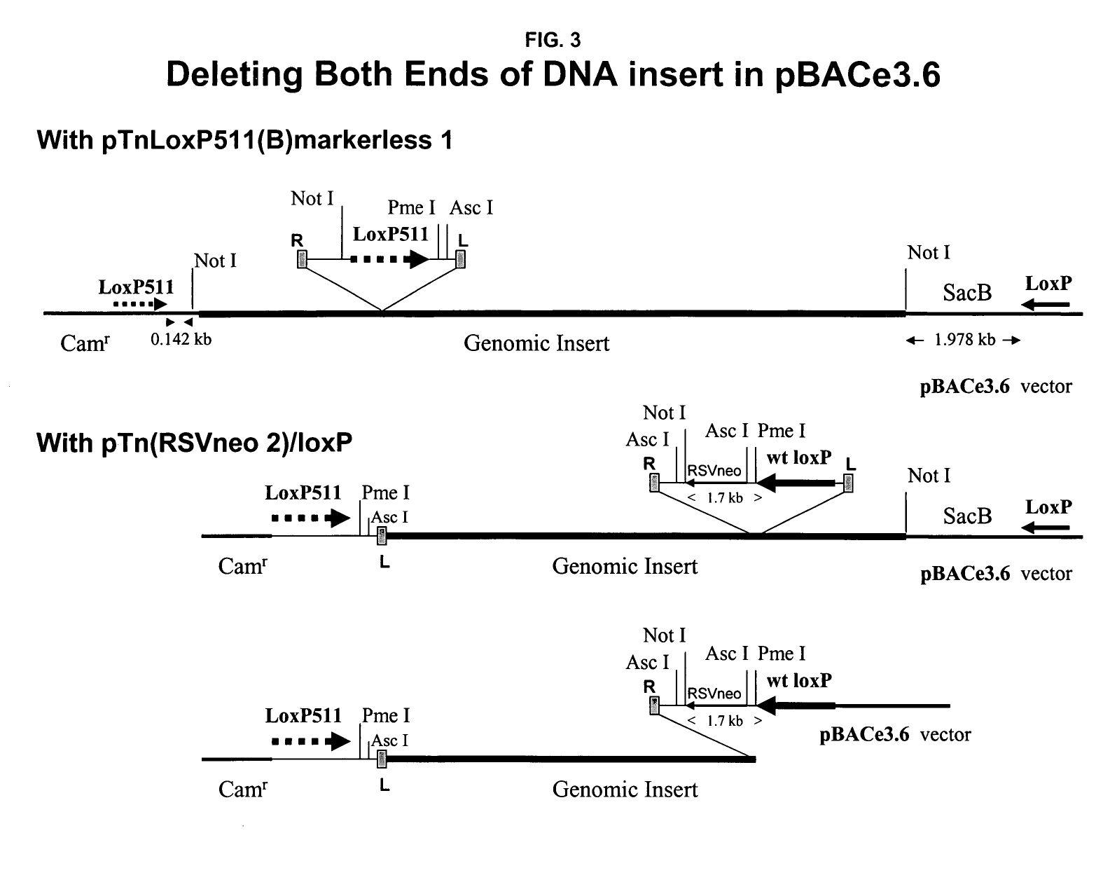

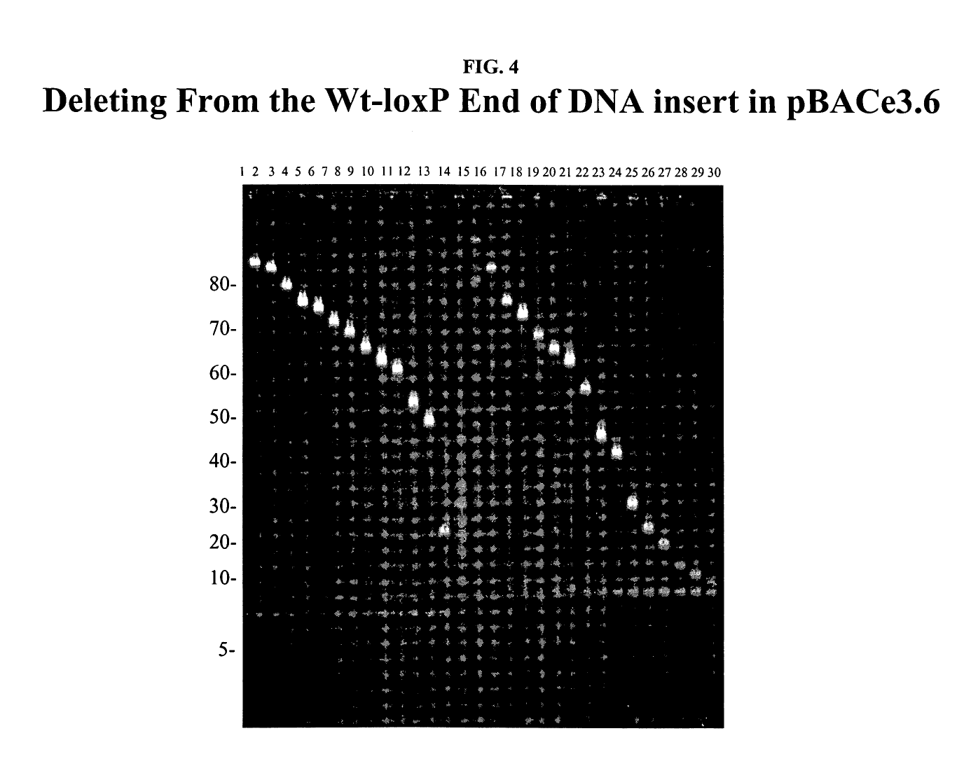

The two NotI sites in pBACe3.6 vector are located asymmetrically with respect to the loxP sites: the NotI sites at positions 2,849 and 11,583 are 1.978 kb and 0.142 kb in front of the wild type and mutant loxP511 sites respectively (35) (FIG. 3). Transposons pTnLoxP511(B)RSVneo 2 and pTn(RSVneo 2)/loxP would substitute the BAC vector NotI site with one from the transposon after recombining the respective loxP sites (FIGS. 1 and 3). Because the NotI sites in both transposons are 1.7 kb away from their respective loxP sites, they would yield BAC-vector DNA NotI-fragments of different size in clones deleted from the wild type or the mutant loxP511 ends of insert: recombinations with either the mutant 511 or wild type loxP sites in the BAC would produce a 10.5 kb or 8.4 kb vector DNA fragment with NotI respectively (lanes 19-30 and 16-30 in FIGS. 2 and 4, respectively). Note that fragments of 10.5 kb and 8.4 kb did not appear together during a deletion experiment with either of these transposons, demonstrating the exclusivity of the recombination reactions. It corroborates the selectivity observed during the P1 transductions described above. Deletion-end sequencing with Tn-based primers also support the conclusion.

Deletions from the wild type loxP end of insert DNA with pTnMarkerless2, described earlier (28), yield the expected vector DNA NotI-fragment of 6.8 kb (lanes 1-13, FIG. 4). Intra-insert deletions independent of LoxP-Cre recombination are isolated only when using markerless transposons (28), because the probability of isolating one in conjunction with a transposon insertion is low, and can be easily identified by their BAC vector DNA fragment with NotI: it is the same size as the starting BAC, as shown in lane 1 of FIG. 2. Note that markerless transposons cannot be used for second round deletions because the starting clone would be less than P1 headful size (28).

A new wild type-loxP transposon pTn(RSVneo 2)/loxP was used to make deletions from the wild type end of insert DNA, producing the expected 8.4 kb BAC vector DNA fragment with NotI (lanes 16-30, FIG. 4).

Truncating Insert DNA from both ends Sequentially in the same BAC

Trimming either end of insert DNA in pBACe3.6 derived clones was readily achieved using transposons specific for the respective ends. Both ends in the same clone can be deleted similarly except that the second round of deletions must necessarily be performed with a resistance gene marker-containing transposon. Preference as to which end of insert DNA, the wild type or the loxP511, will be truncated first dictates the choice of markerless transposon: pTnMarkerless2 (with wt type loxP, described in ref 28) or pTnLoxP511(B)markerless 1 (FIG. 1).

A second round of truncations with pTn(RSVneo 2)/loxP was made with the largest clone in the deletion series obtained with pTnLoxP511(B)markerless 1 (shown in lanes 3 and 31 of FIG. 2). NotI digested DNA isolated from clones obtained in the second round of deletions is shown in lanes 32-40, FIG. 2. Because the starting clone here was less than 110 kb, both deletions and inversions were isolated in about equal numbers (14, 29). Inversions are shown in lanes 42-48 (FIG. 2).

Similar truncations from both ends were also made by first deleting from the wild type loxP end of inserts with pTnMarkerless2, and then using pTnLoxP511(B)RSVneo 2 to trim the other end of a clone isolated in the first round deletions (data not shown).

Processing a Mixture of BAC Clones Transformed with Transposon Plasmid to Increase Throughput of Making End-Deletions

Multiple BACs can be processed together as a mixture in a single tube to make end-deletions. Because clones tend to have different growth characteristics, this is best achieved by transforming each clone separately with the desired transposon plasmid and growing them to saturation before pooling. As many as 32 BACs were transformed in parallel. Aliquots from 9 such transformed BAC cultures were pooled, and processed for deletions exactly as described earlier (29). For simplicity of sequencing downstream, BACs in a pool were transformed with the same Tn-plasmid. Each deletion was sequenced with primers specific to both ends of the insert DNA remaining after the deletion ie. one from the transposon, the other specific to the opposite unaltered end of insert DNA (SP6 or T7). The latter sequence helped identify the parent from which the deletion was obtained. Analysis of the results indicates although each of the 9 BACs is represented in deletions, the distribution is quite skewed: deletions from clone RP23-444K15 dominate four fold over those from RP11-219A15, RP23-209O22 or RP23-92L23, although all four were equally efficient at producing deletions when processed individually.

Sequencing ends of Deletions with Primers from the Wild Type or loxP511 Transposons

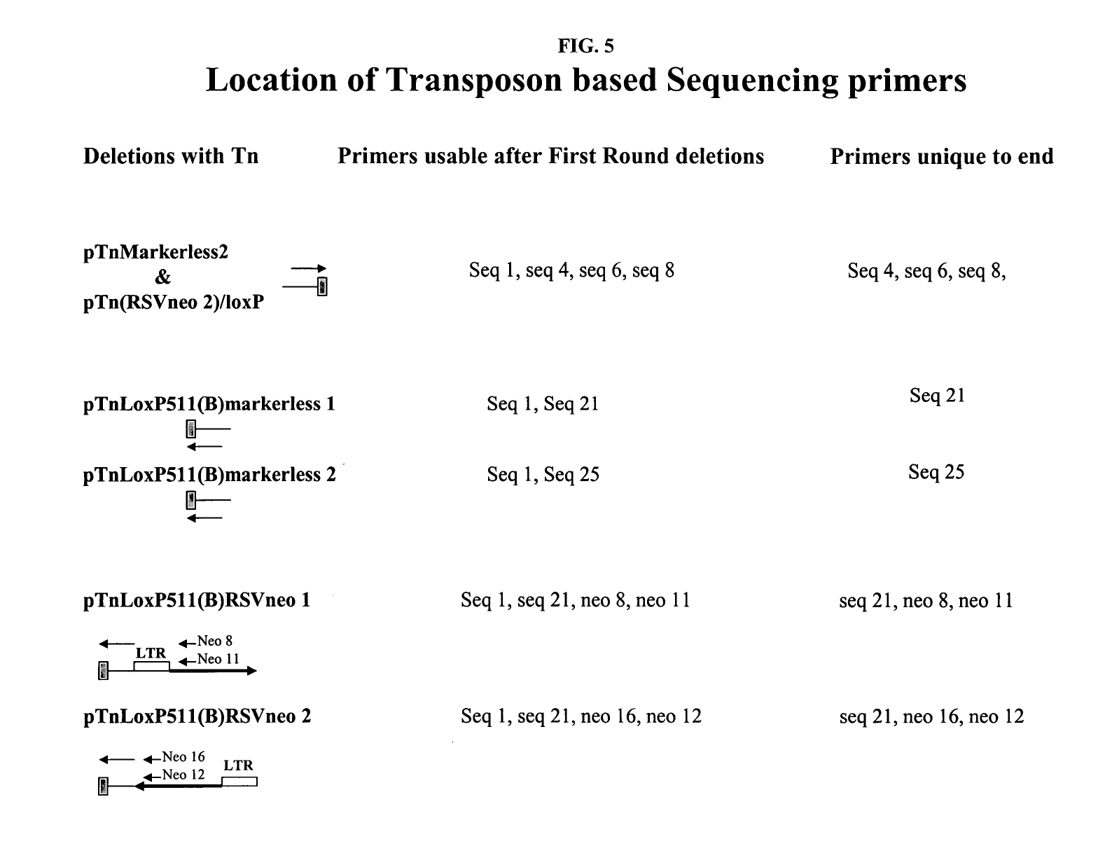

Transposon-ends marked R (pink) and L (green) shown in FIG. 1, 3 and 5 remain after deleting from the wild type or loxP511 end of insert DNA, respectively. Note that the opposite orientation of the loxP511 site in transposons of the “A” series would have left behind the R (pink) end in a deletion, and this would have interfered during end-sequencing of clones after a second round of deletions with the wild type loxP transposon. Because both ends of all transposons described here share the same 70 nucleotides, primer Seq 1 (20) located within it can sequence the new insert-DNA end generated by all of them. However Seq 1 loses its uniqueness after deletions are made from the other end. Primers unique to each end of the transposons described here are listed in Methods. Although both orientations of the loxP511 site and the RSV neomycin gene cassette were constructed in transposons, and analyzed, only those conforming to the desired uniqueness of sequencing primers are presented in FIG. 5 for clarity.

The 600 base reads obtained from each deletion end could BLAST to sequences in the database at the 95-99% homology level. The sequential order of bands in FIGE of the NotI digested DNA from deletion clones corresponded with the order of sequence homology on the chromosome, and BLASTed to only one strand of DNA.

Functionally localizing gene regulatory sequences that operate over large distances using BAC transgenics requires procedures to systematically delete DNA at either end of inserts. Both gap-deletions as well as truncations from one end of the reporter gene-tagged BAC insert have been made either by a targeted deletion strategy using homologous recombination, or using loxP-transposons (2-5). It is believed that the loxP transposon deletion strategy has several advantages over homology-based targeted deletion methods. Induction of recombination often triggers intra-insert DNA rearrangements outside of the regions of homology actually targeted: mammalian DNA is known to be recombinogenic, and is usually kept intact by propagating them in host strains that are rendered highly recombination deficient (6, 7, 36). Occasionally, such intra-insert DNA deletions and/or rearrangements can become excessively more frequent than the desirable targeted deletion (PKC unpublished observations).

A second, more important, advantage offered by the loxP-transposon end-deletion procedure is that an entire array of deletions from a particular end of the BAC insert is obtained in a single experiment. This library of truncated BAC-GFP deletions can be made without the need to construct new targeting vectors each time.

The illustrations in FIGS. 1 and 3 indicate that sequences ahead of a loxP site are retained in the deleted clone. This characteristic can be used to bring regulatory sequences that are far away, or from another gene, to the near vicinity of the gene of interest during truncations. Exogenous sequences, inserted at suitable sites in the loxP transposons, can be easily engineered into a BAC clone to regulate a gene in novel ways.

Studies requiring genes with well defined functional boundaries are likely to benefit from the procedures described here. They allow isolating a gene free of interfering regulatory elements of an adjacent gene existing in the same BAC clone.

The end-deletion technology can be adapted to handle multiple BACs simultaneously. However, results indicate the yield of deletions can be skewed unless BACs with similar growth characteristics are used when pooling.

Recombination of loxP sites with Cre protein has been widely used in both bacterial and mammalian systems (19, 23, 24, 26, 37-41). The potential for using wild type and mutant loxP sites in conjunction is even greater. However, the results from a number of recent studies suggest the exclusivity between the wild type and mutant loxP511 sites might be compromised by the level and persistence of Cre protein (19, 23-26). Results presented here and those from an earlier study (27) indicate that cross-recombination between wild type and mutant loxP sites can be reduced if the Cre protein is transiently expressed as in a phage P1 infection. The differences in specificity observed between results presented here and those reported by other investigators earlier might be related to the different ways by which loxP sites are exposed to Cre protein in vivo. Although removal of host cell factors and mutations in transposases relax target site specificity during Tn10 and yeast Ty1 insertions (42), there is yet no evidence to suggest that additional phage encoded and/or induced factors during a P1 infection alters fidelity of Cre recombinase. Thus in the absence of evidence for enhanced specificity conferred by phage P1 encoded proteins other than Cre, in either bacterial or mammalian systems (43, 44), these results and previous work by others (24, 26) appear to support the idea that high levels and/or persistent exposure to Cre protein might in fact contribute to loxP site promiscuity.

FIG. 1: New Transposon Plasmids Containing Wild Type and loxP511 Sites

In FIG. 1, there is shown a schematic representation of transposon plasmids used for deleting both ends of insert DNA cloned in the pBACe3.6 and related vectors pTARBAC- 1 and -2. The transposon plasmid pTnLoxP511(B)markerless 1, shown in the top panel, has no antibiotic resistance marker within the transposing part marked by the small rectangular boxes colored pink or green. Bottom panel shows a kanamycin resistance gene-containing variant, pTnLoxP511(B)RSVneo 2. The middle panel shows a wild type loxP transposon plasmid with kanamycin resistance gene. Wild type loxP site is shown with the thick continuous arrow, while the mutant loxP511 sites are indicated by bold broken arrows. The pink end R remains in deletion clones generated with the wild type loxP transposons, while the green end L is left behind in deletions with the loxP511 transposons.

FIG. 2: Field Inversion Gel Electrophoresis (FIGE) Analysis of NotI Digested DNA Isolated from Progressive Truncations made from one or both ends of Insert DNA

Referring now to FIG. 2, there is shown at left panel—DNA isolated from deletion clones generated with either pTnLoxP511(B)markerless 1 (lanes 3-15) or pTnLoxP511(B)RSVneo 2 (lanes 19-30) in BAC clone RP23-444K15 and RP 11-219A15, respectively, were analyzed by FIGE. Lane 17 contains the DNA from starting BAC clone RP23-444K15, while lanes 2, 18 and 41 contain 5 kb DNA ladders. Right panel: Second round deletions made with pTn(RSVneo 2)/loxP from the wild type loxP end of insert DNA from the deletion clone shown in lanes 3 and 31. Lanes 32-40 show deletions, while 42-48 show inversions. Deletions with pTnLoxP511(B)RSVneo 2 (lanes 19-30) produce a 10.5 kb BAC vector DNA fragment, while those with pTnLoxP511(B)markerless 1 (lanes 3-15) produce no BAC vector band because one of the NotI sites is lost as a result of the deletion (see FIG. 3). Intra-insert deletions independent of LoxP-Cre recombination produce the same sized BAC vector band as starting BAC (lane 1).

FIG. 3: Schematic Representation of Deletions made Sequentially from the i) Mutant loxP511 end of Genomic DNA Insert Generated with pTnLoxP511(B)markerless 1 (Top Panel) and ii) Wild Type loxP end with pTn(RSVneo 2)/loxP (Bottom Panel)

Referring now to FIG. 3, the transposon is shown as the triangle with the locations of NotI sites indicated. Note that Cre-mediated deletions generated with the loxP511 sites would substitute a NotI with a Pme I site (top panel). Deletions using the wild type loxP sites would retain the NotI site as shown in the bottom panel.

FIG. 4: FIGE Analysis of NotI Digested DNA Isolated from Deletions made from the Wild Type loxP End

In FIG. 4, FIGE analysis show at left panel, DNA isolated from deletion clones generated with pTnMarkerless2 (described in ref 28); in BAC RP23-92L23 is shown in lanes 1-13. Lanes 16-30 shows DNA from first round deletions generated with pTn(RSVneo 2)/IoxP in clone RP23-444K15.Still referring to FIG. 4, lane 15 contains DNA from starting BAC RP23-444K15. Lane 14 shows a 5 kb DNA ladder. The size of BAC vector DNA fragment is 6.8 kb and 8.4 kb with pTnMarkerless2 and pTn(RSVneo 2)/loxP respectively. The starting BAC produces a 8.8 kb BAC vector band (lane 15).

FIG. 5: A Schematic Diagram Showing Sequencing Primers Usable after First and Second Round Deletion

Referring now to FIG. 5, pink rectangles represent the R end of Tn10 remaining in deletions generated with wild type transposons such as pTnMarkerless2 (ref 28), and pTn(RSVneo 2)/loxP. Green rectangles indicate the L end of Tn10 remaining in deletion clones made with any of the loxP511 transposons described in this invention.

Mutually Exclusive Recombination of Wild Type and Mutant loxP Sites in vivo Facilitates Transposon-Mediated Deletions from Both Ends of Genomic DNA in PACs

Materials and Methods

Construction of pJCPAC-Mam2

The oligodeoxyribonucleotides d (GGCCGCATAACTTCGTATAATGTGTACTAT- ACGAAGTTATGTTTAAACGC) (SEQ ID NO:1) and d (GGCCGCGTTTAAACATAACTTCGTATAGTACACATTATACGAAGTTATGC) (SEQ ID NO:20) were annealed to create the mutant loxP*-1 site. The oligodeoxyribonucleotides d (GGCCGCATAACTTCGTATAAAGTAT CCTAT ACGAAGTT- ATGTTTAAACGC) (SEQ ID NO:2) and d (GGCCGCGTTTAAACATAACTTCGTATAGGATACTTTATACGAA GTTATGC) (SEQ ID NO:21) were annealed to create the mutant loxP*-2 site. The two sites loxP*-1 and -2 refer to mutant loxP sites 5171and 2272 respectively, described earlier as better ‘exclusive’ mutants showing efficient recombination in vitro using Cre-containing mammalian cell extracts (21).

A Pme I site was built into each oligodeoxyribonucleotide, and NotI overhangs were generated upon annealing. The dephosphorylated oligodeoxyribonucleotides were ligated into the unique NotI site in pJCPAC-Mam1 (see FIG. 1 of reference 22). The two vectors with a wild type and one of two different mutant loxP sites (loxP*-1 or loxP*-2) in the same orientation and flanking the BamH I site were named pJCPAC-Mam2A and pJCPAC-Mam2B respectively. New libraries of human DNA isolated from a foreskin fibroblast cell line (Viromed, Minnetonka, Minn.) were made in these vectors. Details of the library will be described hereinafter.

Construction of the Transposon Plasmids pTnloxPwt, pTn loxP*-1 and pTnloxP*-2

The markerless transposon plasmid pTnMarkerless2 described previously (28) served as the starting point of pTnloxPwt. The plasmid DNA was linearized at the unique Bgl II site, filled in with Klenow polymnerase, and ligated to the blunt-ended fragment EBNA-ori P used earlier (53). The resulting plasmid is 11 kb in size. Transpositions of pTnloxPwt were selected by P1 headful packaging as described in detail elsewhere (28). All plasmids were propagated in NS3516 cells (laqIq) to prevent activation of the transposase gene.

The transposon plasmid pTnSpliceTerminator (AT) (PKC unpublished results) served as the starting point for both mutant loxP site-containing plasmids. The rabbit B-globin 3′ terminal exon splice acceptor cassette was removed by NotI digestion, and into it was ligated a loxP*-tetracycline resistance gene cassette excised from plasmid pZT344loxP*Tet (J. S. Coren, unpublished data). The two mutant loxP* Tn plasmids contain the chloramphenicol resistance gene and a PGKpuromycin resistance gene on one side of the mutant loxP site, and the tetracycline resistance gene on the other side. The location of the tetracycline resistance gene with respect to the mutant loxP site ensures tetracycline resistance only in inversions generated from the transposition events. Both deletions as well as inversions with pTn loxP*-1 and pTnloxP*-2 were therefore selected using the chloramphenicol resistance marker.

Generation of Nested Deletions in Individual PAC Clones

Clones with large inserts were identified in the pilot libraries generated in pJCPAC-Mam2A and pJCPAC-Mam2B by FIGE after NotI digestion. Nested deletions using pTnloxPwt were generated in several clones as described previously (28). Deletions with pTnloxP*-1 and pTnloxP*-2 that contain an antibiotic resistance marker to score for transpositions were generated as described previously (29).

End-Sequencing of PAC Deletion Clones

Typically, miniprep DNA was isolated from 60 clones picked randomly from the several hundred to a thousand member PAC deletion library. Of these, approximately 60% were of unique size on FIGE. The DNA of 20 clones from each deletion series was sequenced directly using a transposon-end primer (20) and Big Dye Terminator chemistry on an ABI-3100 AVANT genetic analyzer. Primer extended products were purified using Magnesil (Promega Corporation) according to procedures supplied by the manufacturer and described previously (55). New primers used to sequence the newly created ends of deletions from the mutant loxP side of insert DNA already trimmed from the wild type loxP-end are listed below:

| | | |
|---|---|---|
| Seq 11 d | (CTTCCATGTCGGCAGAATGC) | (SEQ ID NO:3) |
| Seq 12 d | (GTTCATCATGCCGTCTGTGATG) | (SEQ ID NO:4) |
| Seq 13 d | (CGCTGGCGATTCAGGTTCATC) | (SEQ ID NO:5) |
| Seq 14 d | (CAAGGCGACAAGGTGCTGATG) | (SEQ ID NO:6) |

Results

Construction of PAC Clones with Human DNA Inserts Flanked by Wild Type and Mutant loxP Site

The PAC cloning vector pJCPAC-Mam1 described previously (22) was linearized at its unique NotI site and ligated to two versions of a mutant loxP site to generate pJCPAC-Mam2A and pJCPAC-Mam2B as described in Materials & Methods. PAC libraries of 80-140 kb size-selected human genomic DNA were constructed in these shuttle vectors and details will be reported elsewhere (J. S. Coren manuscript in preparation).

Analysis of Recombination of Wild Type and Mutant loxP Site-Containing Plasmids in vivo using Phage P1 Transduction

Several studies have analyzed recombination between wild type and mutant loxP sites with Cre protein using both non-native systems in cells (23, 33, 24) and in vitro protocols with mammalian cell extracts containing Cre protein (21), with mixed results. Certain mutant loxP sites such as loxP 511 and loxP 512, as well 5171 and 2272 (referred here as loxP*-1 and -2 respectively), produced conflicting results in these studies. Success in creating deletions specific to each end of insert DNA in BACs and PACs in a controlled manner requires that there be no leakage or crossover recombination between wild type and mutant loxP sites. The gel-based assay using ethidium bromide stain to detect product DNA bands of recombination in (25) allows at best only a 20 fold difference in signal to be registered. It was therefore essential to test recombination cross-reactivity by an assay with greater sensitivity and broader range, and that also used conditions specifically developed for making progressive deletions from one end of insert DNA (14). Recombination analysis was therefore extended to include transduction using phage P1.

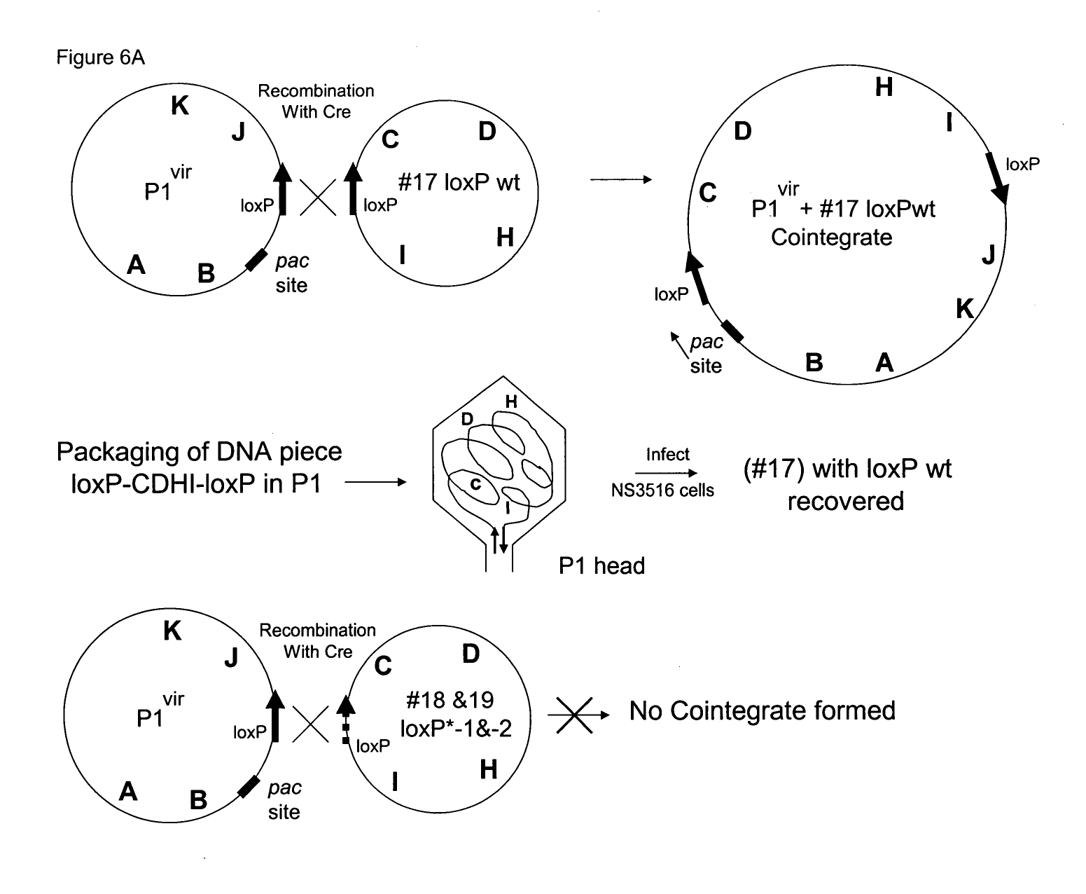

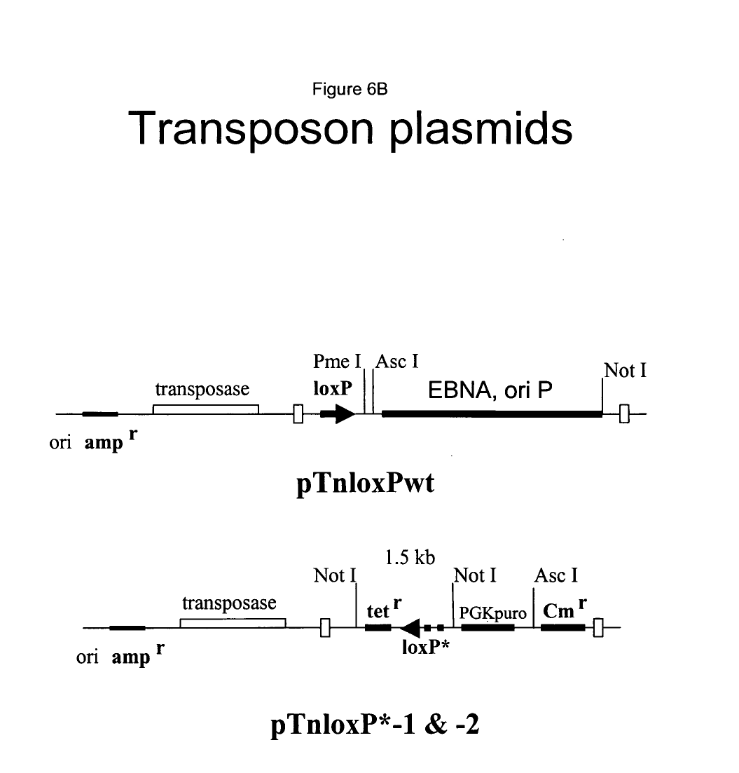

The linear DNA within viable phage P1 is flanked by two copies of wild type loxP sequence. P1 is also known to transduce with high efficiency a second plasmid with a wild type loxP site (30-32). Its ability to transduce the Tn plasmids that carry either wild type or mutant loxP sites was therefore tested to evaluate recombination cross-reactivity between these sites (see FIG. 6A for illustration). The three Tn plasmid-containing strains #17 with wild type loxP, #18 with mutant loxP*-1 and #19 with mutant loxP*-2, were each infected with P1 vir phage, and the cell-lysates were used to infect fresh NS3516 cells using procedures described previously (29). The NS3516 cells containing the phage-transduced Tn plasmids were selected in LB plates containing antibiotics specific to the Tn plasmid. The results shown in the top 3 rows of Table I clearly demonstrate that phage P1 vir is capable of transducing only the wild type loxP site containing plasmid #17. Cross-recombination between the wild type loxP site in P1 and either loxP*-1 in #18, or loxP*-2 in #19, that would have had to occur for their recovery, was found to be at least 1000 fold lower. Using a larger cell sample and multiple plates to compare relative efficiencies of transduction, we estimate that wild type loxP sites recombine with one another at least 10,000 fold more efficiently than with either of the two mutant sites under these native in vivo conditions of transient Cre protein expression during P1 infection.

Generating Progressive Deletions from the Mutant loxP end of DNA Inserts with a Transposon Containing the same Mutant loxP Site

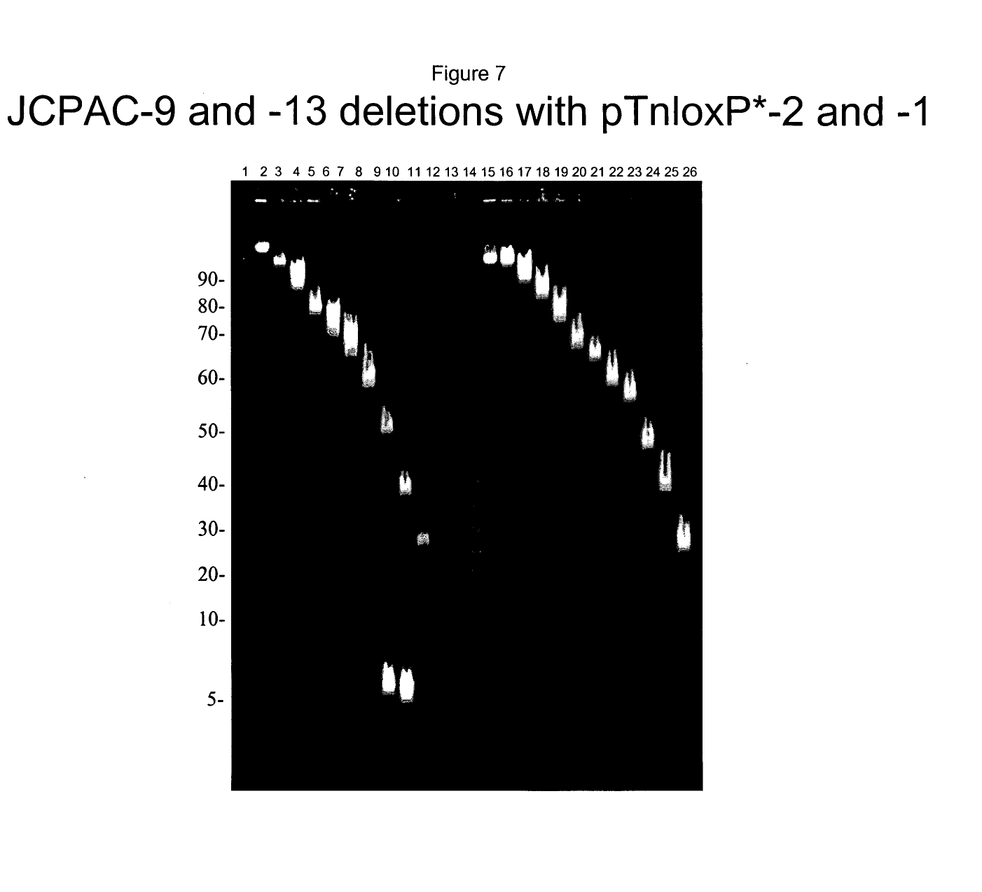

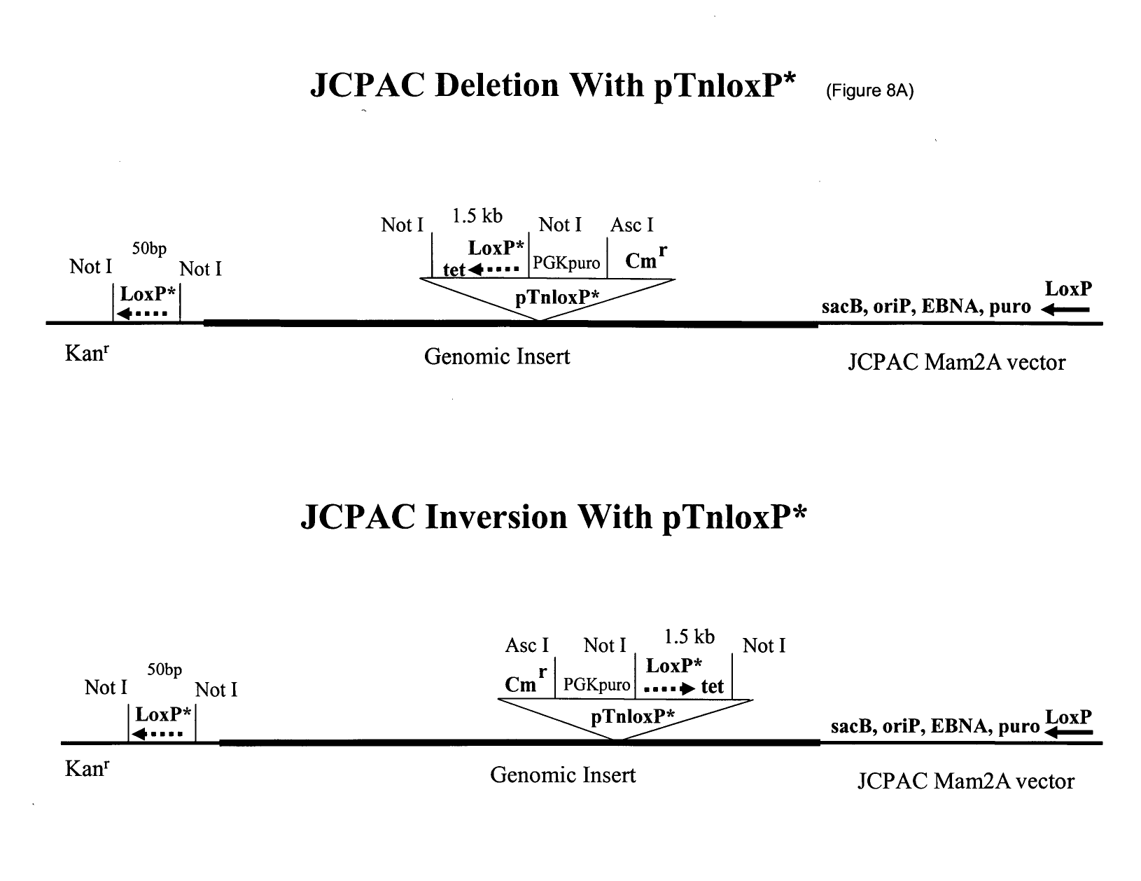

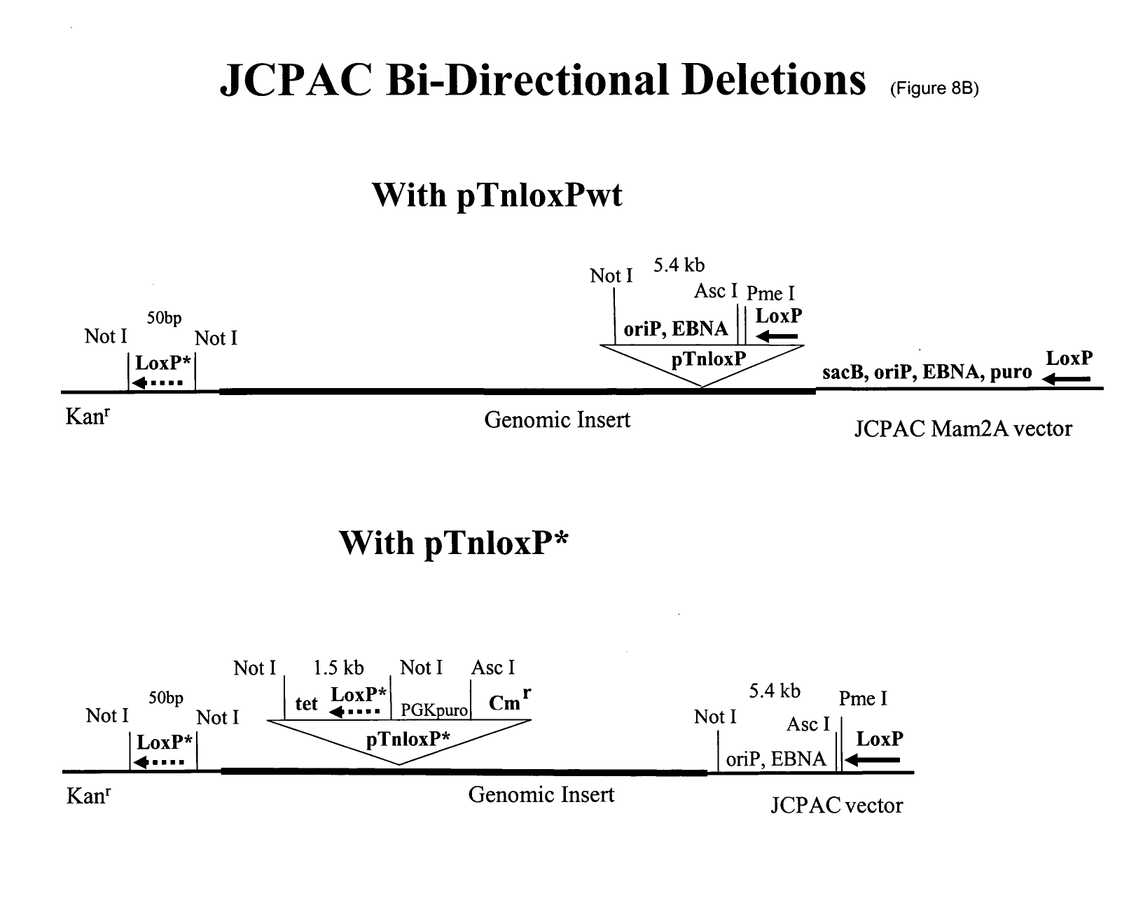

New transposon plasmids pTnloxP*-1 and pTnloxP*-2 (shown schematically in FIG. 6B) were constructed as described in Materials and Methods, and were tested initially on several clones from the genomic libraries constructed in pJCPAC-Mam2A and pJCPAC-Mam2B. Detailed analyses on two clones JCPAC-9 and JCPAC-13, with 120 kb and 140 kb DNA inserts respectively, are presented here. Thus plasmids pTnloxP*-1 and pTnloxP*-2 were introduced into JCPAC- 13 and JCPAC-9 respectively, and transposition induced with IPTG as described previously (29). Transduction of the resulting deletions with P1 phage was considered not to be a hurdle as the PAC vectors contained a wild type loxP site in addition to the mutant one. The results are shown in FIG. 7, and schematic illustrations provided in FIG. 8A (see also FIG. 1 of refs 14 and 28). Because the starting PAC clones have a NotI site only at one end of the insert, digestion of their DNA with NotI enzyme produce no separate vector band (lanes 2 and 15). Deletions generated in JCPAC-9 and JCPAC-13 with pTnloxP*-2 and pTnloxP*-1 exchanges one of the NotI sites in the PAC vector with one from the transposon (FIG. 8A). Thus, no separate vector DNA band is seen in deletions either (lanes 3-11 and 16-26, FIG. 7).

A set of deletions from either PAC was sequenced directly using a transposon-end based primer (20). All deletions produced sequence reads that scored between 95 and 99% identity exclusively with one strand of the genomic DNA insert by BLAST analysis: JCPAC 9 and 13 deletions mapped to human chromosome 1 and 4 respectively. The end-sequences also matched an order consistent with the size of the deletion clone in the array (data not shown). Taken together the results therefore validate that deletions generated with the mutant loxP transposons truncate exclusively from the mutant loxP end of the insert DNA in the PAC clone. Deletions were made in four additional PACs with inserts mapping to chromosomes 10, 15, and 19 from the two libraries (a pair from each of the two libraries with inserts flanked by wild type and either loxP* 1 or loxP*2) with identical results.

Closer inspection of lanes 9 and 10 of FIG. 7 reveals DNA at the position of the 5 kb marker band, reminiscent of that from transposon plasmid DNA (20). Further analysis of these two deletion clones revealed that they were both ampicillin and tetracycline-resistant, indicating that the mutant loxP transposon plasmid was indeed transduced by phage P1. This is contrary to what one might predict from the results discussed in section 3.2, and is addressed herein later.

A Mutant loxP Plasmid is Efficiently Transduced with Phage P1 if a Second Plasmid in the Cell has both a Wild Type and the same Mutant loxP Site

It is plausible that the recovery of mutant loxP plasmids by P1 phage seen in lanes 9, 10 of FIG. 7 is due to a piggy-backing phenomenon. The following experiment tests the hypothesis. Unlike the experiments described in Section 3.2, P1 transduction of mutant loxP plasmids was conducted this time around in the presence of a second large plasmid containing, in addition to a wild type loxP site, i) a mutant lox P site of the same kind, ii) a mutant loxP site of a different kind iii) a mutant loxP site of the same kind but not as part of the same DNA molecule. The transposase gene was kept repressed in these experiments, and characterization of plasmid DNA isolated from transductions confirmed that no transpositions occurred. The results are shown in Table 2.

Three different types of PAC plasmids were used: Clones 2 and 5 contained only a wild type loxP sequence while 8, 9 and 12, 13 carried a wild type and one of two mutant loxP sites flanking the insert DNA (see Table 2 for descriptions). The first member of each pair (2, 8 and 12) was less than P1 headful size, while the second member (5, 9, and 13) is larger than 110 kb. Phage P1's ability to transduce each of these PAC plasmids was tested (FIG. 6A). Results in Table 2, column 5 are in accordance with those described earlier. Namely, plasmids smaller than the capacity of a P1 head are efficiently transduced, while those larger than this limit size are not (14).

The ability of the three pairs of PAC plasmids to piggy-back the Tn plasmids #17, 18, and 19 upon transduction with phage P1 was next tested. Each Tn plasmid contains either a wild type or a mutant loxP site, and was transformed into each PAC clone. Transformed cells were selected on LB agar plates containing either kanamycin plus chloramphenicol (#17) or kanamycin plus tetracycline (#18 and #19). Single transformed colonies were expanded for experiments. Table I shows results of three independent experiments using different single colonies.

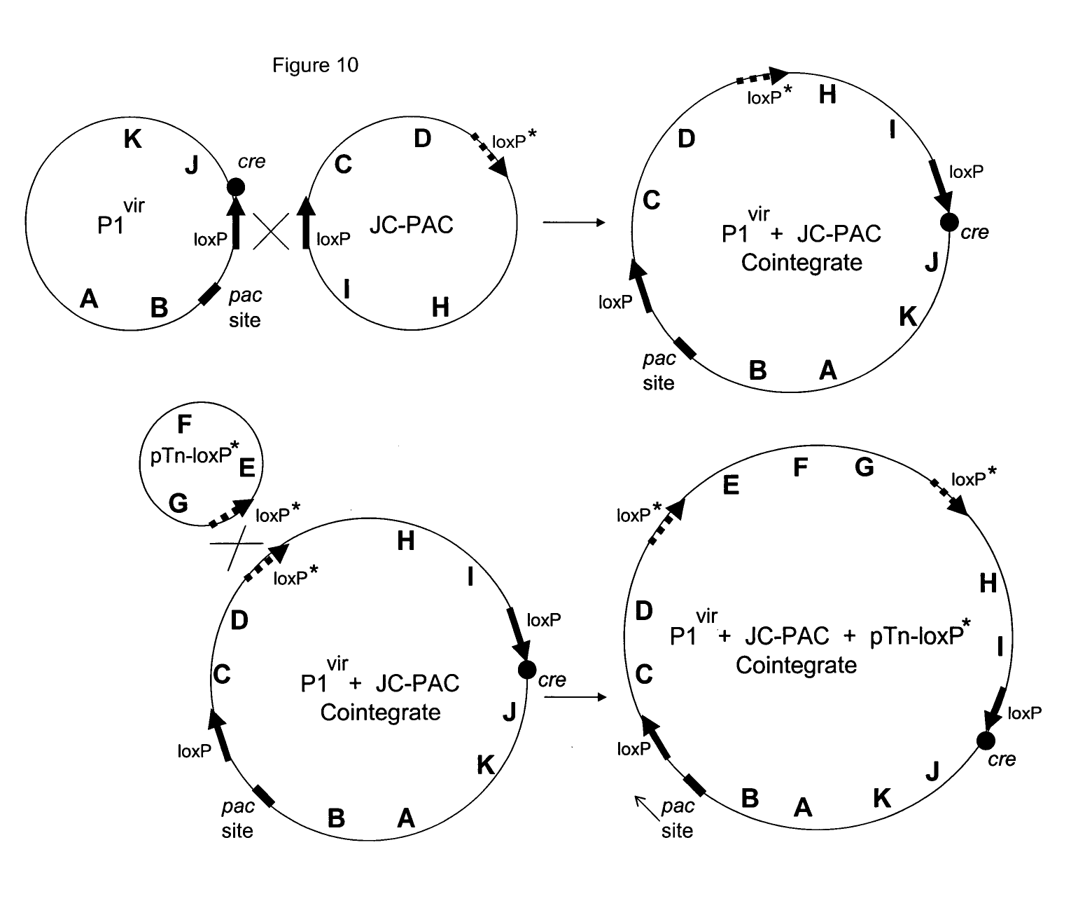

Although P1 could not transduce a mutant loxP plasmid directly (section 3.2), it can do so efficiently if there is another plasmid in the cell that contains both a wild type and the same mutant loxP site (illustrated in FIG. 10). This is seen for both 8/19 and 12/18 in Table 2. Cross recombination between loxP*-1 and -2 is non-existant, as shown by the results of 8/18 and 12/19. Piggy-backing requires not only the same mutant site on both PAC and Tn plasmid, but occurs even when the PAC is too large to be transduced itself (compare results of 9/19 and 13/18 with 8/19 and 12/18). The efficiency is lower in that case. The exquisite recombination specificities of mutant and wild type loxP sites seen with the native in vivo conditions used here are contrary to earlier reports (23, 24).

The requirement that the mutant and wild type loxP sites be part of the same molecule (point # iii)) is demonstrated by the fact that PAC clones 2 and 5 with wild type loxP are incapable of helping P1 phage transduce mutant loxP plasmids 18 or 19 (see results of 2/18 and 2/19), despite both 18 and 19 existing in the cell at 50-100 copies owing to their pBR322 origin of replication.

Generating Deletions from both Ends of Insert DNA in Two PAC Clones

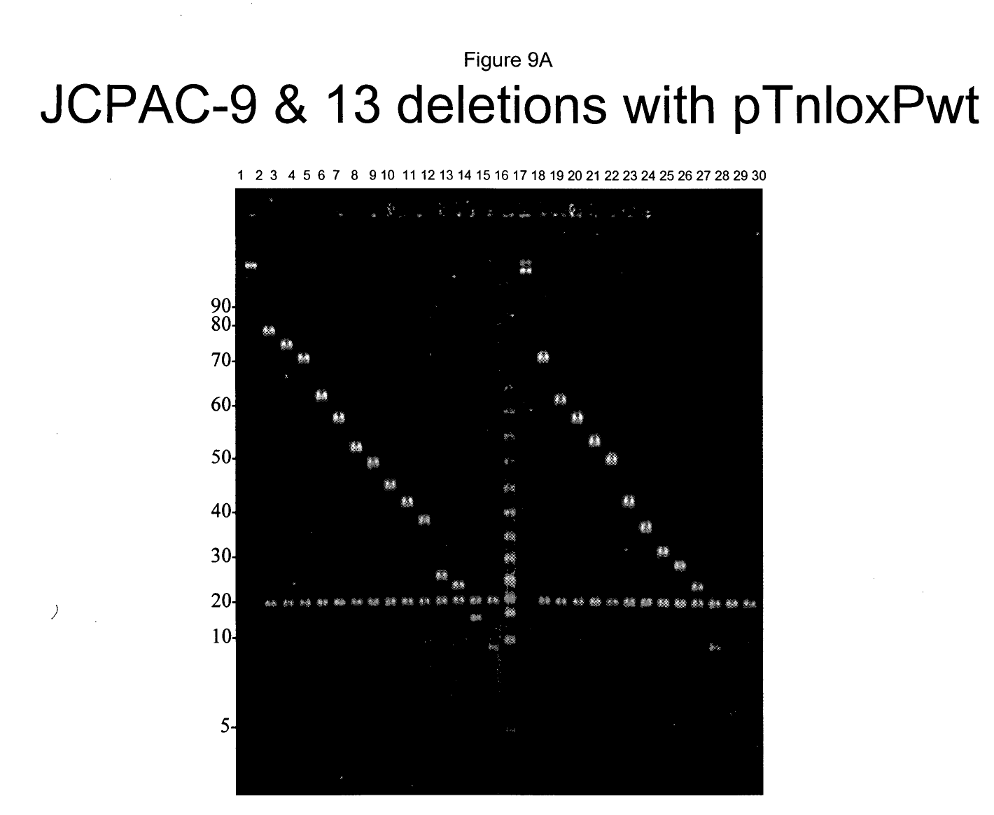

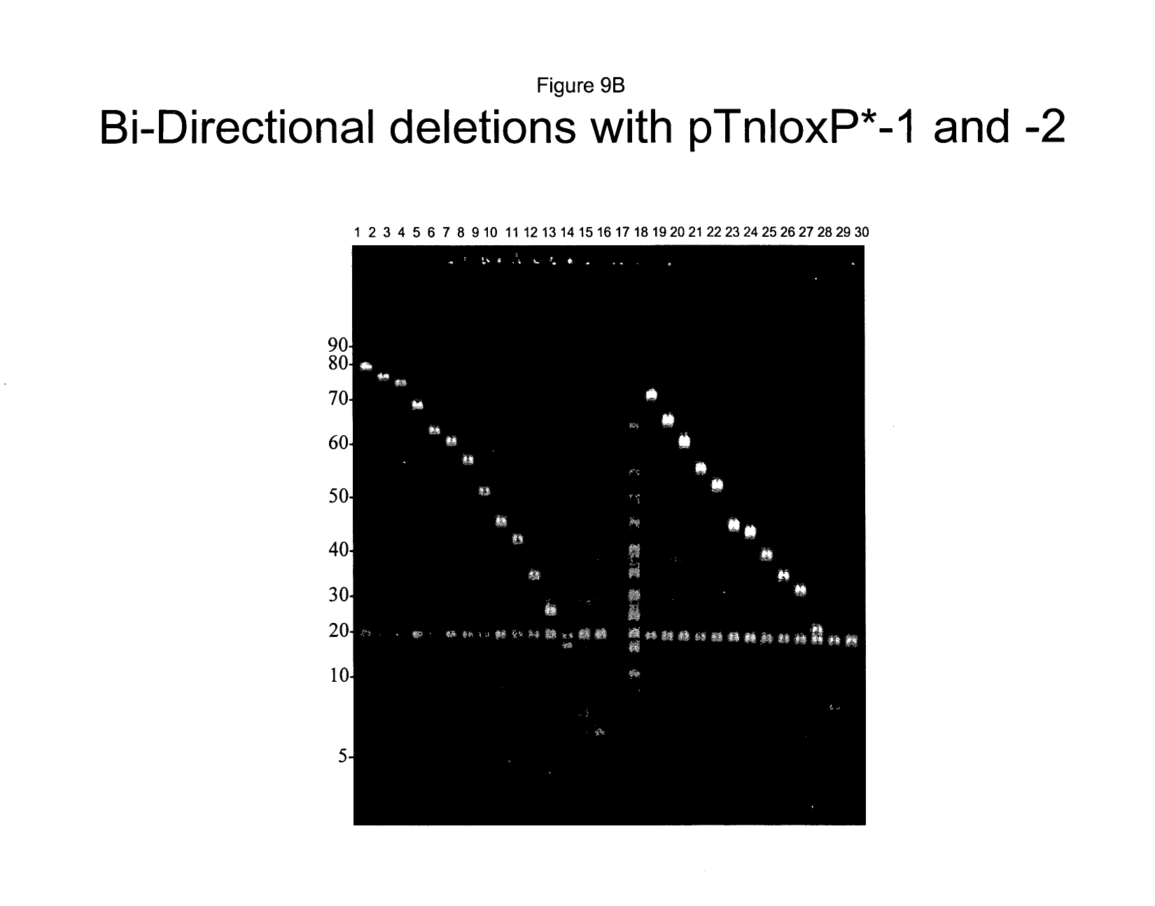

Having established that recombination between wild type and mutant loxP sites by Cre protein is mutually exclusive, attention was refocused on generating bi-directional deletions of insert DNA in the PAC clones. Deletions from the wild type loxP end of the genomic inserts in JCPACs 9 & 13 were made first for the following reason: transpositions of markerless transposons such as pTnloxPwt are selected by the ability of the P1 head to package both loxP sites from the deleted cointegrate, and requires a larger than P1 headful length (110 kb) starting PAC clone (28). Deletion libraries were generated in JCPACs 9 & 13 with pTn loxPwt using procedures identical to those described earlier (25, 26). A NotI digest of several clones from each deletion series is shown in FIG. 9A. As shown schematically in FIG. 8B, a deletion using pTnloxPwt introduces a NotI site in the clone such that the insert DNA is now flanked on either side by a NotI site. Thus NotI digest of deletion clone DNA generates a 20 kb vector DNA band not seen in digests of starting PAC clones or any intra-insert deletions generated by illegitimate recombinations (see ref 28 for discussion). Deletion-end sequencing of several clones, and BLAST analysis revealed homology of all deletions from the same PAC clone to only one strand of DNA, and arrayed according to their sizes.

The largest clone from each series was chosen for creating bi-directional deletions, and the results are shown in FIG. 9B. Thus clones 1 and 77 derived from JCPAC clones 9 and 13 respectively, were subjected to the nested deletion procedure this time using the respective mutant loxP transposons. Plasmids pTnloxP*-1 & -2 were introduced into deletion clones 77 and 1 respectively, and deletions were generated using procedures identical to those described previously (29). NotI digests of DNA from several clones from either series is shown in FIG. 9B. The locations of NotI and loxP sites in PAC and transposon (shown in FIG. 6B and FIG. 8B) predict no change in the vector DNA band between starting and deletion clones. A sizable fraction of isolated clones in the second step were inversions generated by transpositions of the mutant loxP site in an orientation opposite to that in the PAC. Inversions were isolated this time around because the starting deletion clones 77 and 1 are less than P1 headful size. Such clones were tetracycline resistant (see FIG. 8A) and easily identified.

Sequencing Ends of Insert DNA Truncated at Both Termini with Transposon Based Primers

While the first round of deletions from either the wild type or mutant loxP end can be sequenced with transposon primers described earlier (19), ends of inserts deleted from both ends required new primers from transposon regions that are unique to the newly created end. Thus primers from within the chloramphenicol resistance gene specific to mutant loxP transposons were used to sequence the new ends of insert DNA and are described in Materials and Methods. All chloramphenicol related sequences are deleted from pTnloxPwt used for truncating the other end.

Discussion

The ability to truncate large pieces of DNA from both ends in a controlled manner should facilitate mapping gene regulatory sequences that act over large distances so as to be able to functionally define their boundaries, and make available entire genes in their chromosomal contexts for numerous applications. The highly specific recombination demonstrated by wild type and the two mutant loxP sites with wild type Cre protein under native in vivo conditions might become useful in the design of sequence selective tags and/or molecular switches in certain applications. Using a phage P1 transduction assay to evaluate relative recombination efficiencies between wild type and either of the two loxP mutant sites, a much higher degree of specificity has been demonstrated than was possible earlier with the gel based assay (21). Also, the piggy-backing of mutant loxP plasmids during P1 transduction described in Section 3.4 offers a unique way to measure cross recombination between pairs of mutant loxP sites without having to construct new phage carrying those sites. The differences between the results obtained in vivo here and those reported previously (23, 24) might be related to the transient versus constitutively expressed Cre protein available for the recombinations. LoxP site promiscuity does appear to increase with the level and persistence of Cre protein. Recombining a pair of either wild type or the two mutant loxP sites with similar efficiency by Cre protein expressed during P1 infection is not surprising. The most sensitive sites on DNA with respect to Cre protein recognition, base pairs 2 and 6, lie within the 13 bp inverted repeats of the loxP site (56), and these are invariant in all the loxP sites tested here. The mutations are in only the 8 bp spacer region.

The selectivity between wild type and mutant loxP sites is also not surprising as this region is thought to require complete base pairing during cross-over (25). This is consistent also with efficiencies of homologous recombination being highly sensitive to mutations in the region of homology between partners: the 8 base spacer of loxP apparently serves a similar role to the spacer region in the FLP/FRT system and presumed Holiday-like junctions would otherwise contain disruption of base pairing (57).

The results of Table 2 demonstrate that phage P1's ability to transduce a mutant loxP plasmid depends upon a third plasmid having both a wild type and an identical mutant loxP site. Such a scenario suggests that multi-plasmid cointegrates are bona fide intermediates in the cell. A schematic representation of such intermediates likely to participate in the transduction process is shown in FIG. 10. Packaging starts at the pac site and goes clockwise (indicated by the thin arrow) till the P1 head is full. Note that the DNA piece BAKJ in the final co-integrate is phage DNA, and is lost upon packaging. If the DNA piece HI (genomic insert) is small as in PAC clones 2, 8 and 12, the second wild type loxP site (between I and J) is able to fit comfortably inside the PI head (see FIG. 1 of ref 14). Cre protein is expressed immediately upon entry of the linear DNA into the cell upon infection, and helps recombine the DNAs between the two mutant and wild type loxP sites. Both plasmids are therefore regenerated, and both antibiotic resistance markers are expressed as seen for clones 8/19 and 12/18 in Table 2.

If the DNA piece HI is very large as in PAC clones 5, 9 and 13, the second wild type loxP site (between I and J) is unable to fit in the P1 head. Upon infecting fresh NS3516 cells, the DNA inside the P1 head is now unable to salvage the starting PAC clones 5, 9 and 13 (see FIG. 1 of ref 14). However the two mutant loxP sites recombine to regenerate, albeit a 10-20 fold lower efficiency, the mutant loxP plasmids as seen for clones 9/19 and 13/18 in Table I. Note that newly expressed Cre protein would not be available in this case as the gene encoding it is lost while packaging of the DNA in the P1 head, and the occasional recombination of mutant loxP sites most likely is mediated by cellular recombinases.

FIG. 6A: A Schematic Representation of P1 Transduction of Tn Plasmids pTnloxPwt, pTnloxP*-1 and pTnloxP*-2

As shown in FIG. 6A, the phage DNA circularizes upon entering the cell by Cre recombination of its terminally redundant loxP sites, and forms cointegrates with the transposon plasmid if the loxP site carried by it is also wild type. Still referring to FIG. 6A, cointegrate DNA is cleaved at the “pac site”, and packaging occurs in the direction shown by the thin arrow adjacent to the “pac site”. The transduced plasmid is recovered when the phage containing Tn plasmid DNA infects new NS3516 cells. Mutant loxP site-containing Tn plasmids pTnloxP*-1 and -2 fail to form cointegrates with the wild type loxP site-containing phage DNA under identical conditions. The recovery of the mutant loxP site containing transposon plasmids, if any, is taken as a measure of cross-recombination activity between mutant and wild type loxP sites.

FIG. 6B: New Transposon Plasmids Containing Wild Type or Mutant loxP Sites

In FIG. 6B, a schematic representation of the transposon plasmids constructed for bi-directional deletions is shown. The transposon plasmid pTnloxPwt has no antibiotic resistance conferring marker within the transposing part of the DNA marked by the small rectangular boxes. The wild type and mutant loxP sites are indicated by bold and broken arrows respectively.

FIG. 7: Progressive Deletions from the Mutant loxP End of Genomic DNA Insert Generated with pTn loxP*-1 and pTnloxP*-2

Reference is now made to FIG. 7, wherein DNA isolated from deletion clones generated with either pTn loxP*-1 or pTnloxP*-2 in clones JCPAC-13 and 9 respectively, were analyzed by FIGE after digestion with NotI enzyme. Lanes 2 and 15 show the starting JCPAC-9 and 13 respectively. Lanes 1 and 14 contain 5 kb ladder as size standard.

FIG. 8A: Schematic Representations of Deletions and Inversions Formed with pTnloxP*-1 and-2 in JCPAC-13 and 9

The transposon is shown as the triangle with the locations of NotI sites indicated. Note that inversions are isolated only if the starting PAC plasmid is less than 110 kb.

FIG. 8B: Schematic Representations of Sequential Deletions Generated First with pTnloxPwt and then with either pTnloxP*-1 or -2 in JCPAC-13 and 9 Respectively

Note that all of the EBNA, oriP, and PGK-puro gene cassettes are replenished after the bi-directional deletions so as to render the deletion clone effective in mammalian cells.

FIG. 9A: Deletions from the Wild Type loxP End Generated with pTnloxPwt in JCPAC-9 and-13

NotI digests of deletion clone DNA analyzed by FIGE. Lanes 1 and 17 contain starting JCPAC-9 and - 13 DNA respectively. Lane 16 shows a 5 kb ladder.

FIG. 9B: Deletions from the Mutant loxP Site End of Deletion Clones 1 and 77 Generated with pTnloxP*-2 or -1 Respectively

Deletion clones 1 and 77 were obtained earlier from JCPAC-9 and -13 respectively by deleting with pTnloxPwt. FIGE analysis of NotI digested clone DNA. Lanes 1 and 18 contain the starting deletion clones 1 and 77, and lane 17 shows a 5 kb ladder.

FIG. 10: A Schematic Diagram Showing Possible Multi-Plasmid Co-Integrates as Intermediates during Transduction of Mutant loxP Site-Containing Plasmids by Phage P1

The terms ‘cre’ stands for the gene encoding Cre protein and is indicated by the black dot, ‘pac site’ represents a sequence recognized by the packaging machinery of P1 where a double strand cut is made and packaging of DNA starts. The thin arrow adjacent to the ‘pac site’ indicates the direction of packaging.

Selecting Transpositions Using Phage P1 Headful Packaging: New Markerless Transposons for Functionally Mapping Long Range Regulatory Sequences in BACs and PACs.

Materials and Methods

Primers used for Amplifying the Kanamycin Resistance Gene from pACYC177

A primer pair was designed from sequences flanking the entire kanamycin resistance gene from plasmid pACYC177 (GenBank Accession # X06402). The forward primer had Asc I and NotI restriction site sequences built into its 5′end, while the reverse primer had only Asc I site at its 5′ end. The PCR amplified product was 1044 bp in length and contained sequences 1768-2791 from pACYC177. The sequences of the two primers were as follows:

| | |
|---|---|
| kanF | |
| 5′ GGCGCGCCGCGGCCGCGTGATCTGATCCTTC 3′ | (SEQ ID NO:7) |
| kanR | |
| 5′ GGCGCGCCGTCAAGTCAGCGTAATGC 3′ | (SEQ ID NO:8) |

The two primers have different combinations of built-in restriction sites so as to enable placing the NotI site asymmetrically in the loxP-transposon. This ensures that the vector band obtained upon NotI digestion of DNA from a deletion clone generated with this transposon has a distinguishable size on field inversion gel electrophoresis (FIGE).

Construction of pTnMarkerless1 and pTnKan Transposon Plasmids

Amplifying Kanamycin Resistance Gene Fragment from pACYC177

Plasmid pACYC177 DNA from clone E4151 S (New England BioLabs) was isolated using the alkaline lysis miniprep procedure (19). A 1044 bp fragment was PCR amplified with AmpliTaq DNA polymerase from Perkin Elmer using primers kanF and kanR and pACYC177 DNA as template. Each reaction of 25 μL, contained 1× Reaction buffer (Perkin Elmer), 1.5 mM magnesium chloride, 10 ng of DNA template and 15 picomoles of each primer. A cycle of 1 minute denaturation at 95° C. followed by 1 minute annealing at 60° C. and 90 seconds elongation at 72° C. was used for 35 cycles. The sample was initially denatured for 3 minutes at 95° C. A fifth of the product was analyzed on 1% agarose 0.5× TBE gels.

Cloning Amplified DNA Fragment in TA Cloning Vector pCR2.1

The PCR amplified DNA was cloned into pCR2.1 (Invitrogen corporation, Carlsbad, Calif.) using the TA cloning kit manufacturer recommended procedures. Subdloning was performed to facilitate restriction digestions with Asc I. Cleavage with enzymes are sometimes incomplete when nucleotide overhangs are too small adjacent to a restriction site. White colonies were selected on LB agar plates containing kanamycin. Alkaline lysis minipreps were made from several colonies and the DNA analyzed after digestion with Asc I and NotI separately. Clones that were linearized with NotI and produced the 1044 bp band upon Asc I digestion were selected.

Creating pTnMarkerless1 from pTnBAC/loxP

The RSV neomycin gene cassette was removed from pTnBAC/loxP by digesting plasmid DNA with Asc I, and religating. Several clones were picked, the plasmid DNA isolated and tested for the presence of Asc I, Pme I, and Bam HI, and absence of the NotI site. The resulting plasmid was named pTnMarkerless1 because it contains no bacterial antibiotic resistance gene within the transposable part of its DNA: the RSV neomycin gene that conferred kanamycin resistance in bacteria and neomycin resistance in mammalian cells is removed (FIG. 1A). BAC/PAC clones transformed with this plasmid were selected on LB agar plates containing 120 μg/mL of ampicillin. The gene for ampicillin resistance is outside the transposon boundaries.

Creating pTnKan from pTnMarkerless1

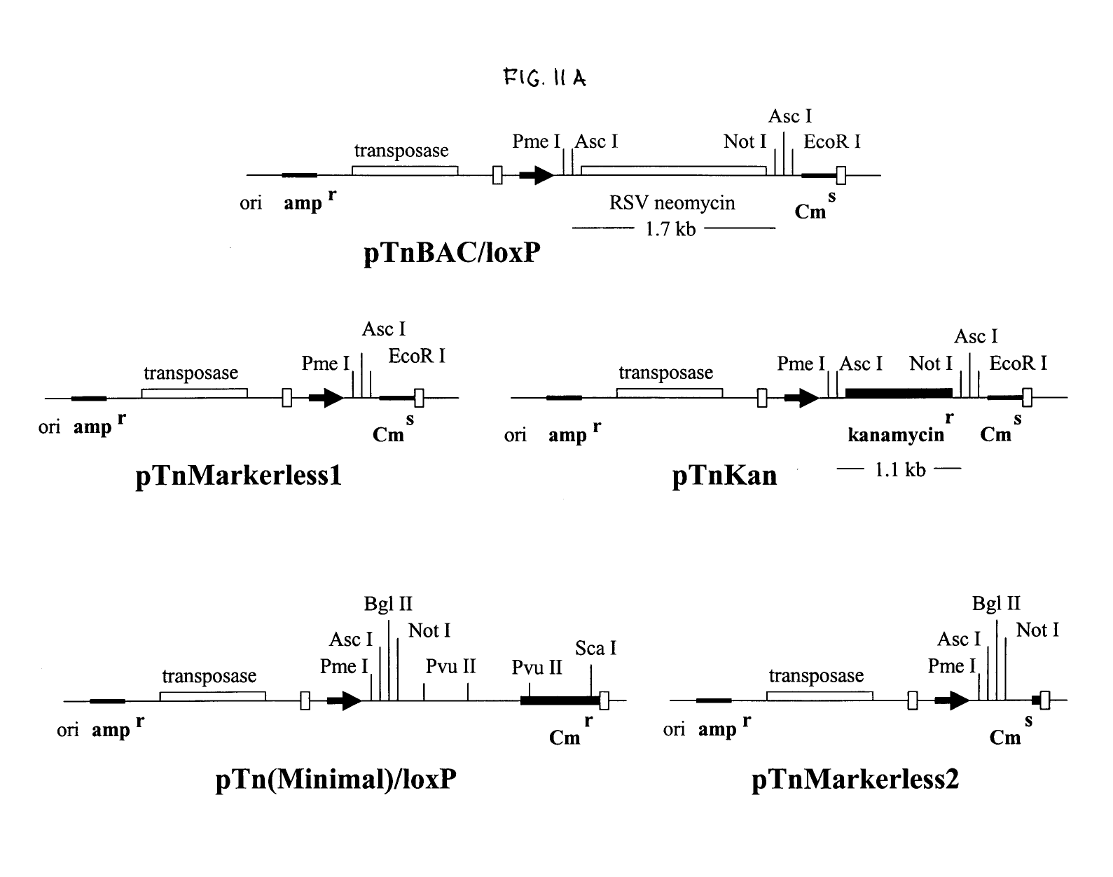

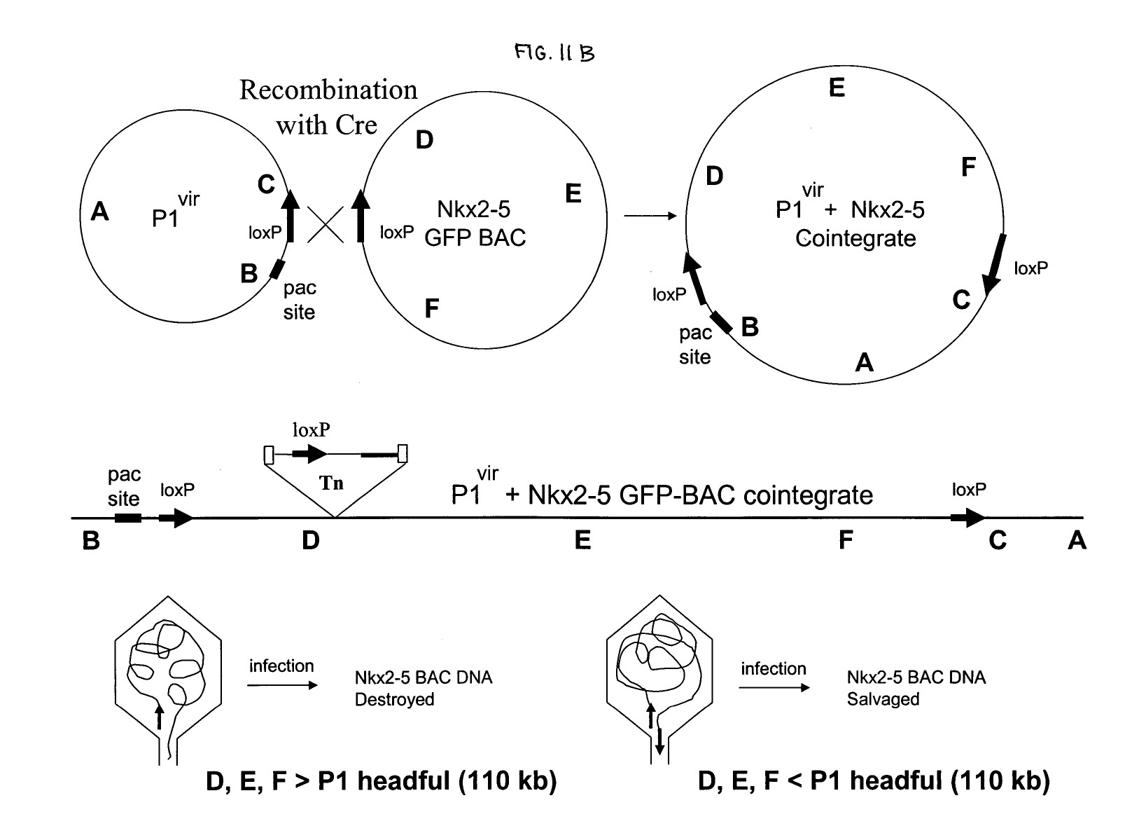

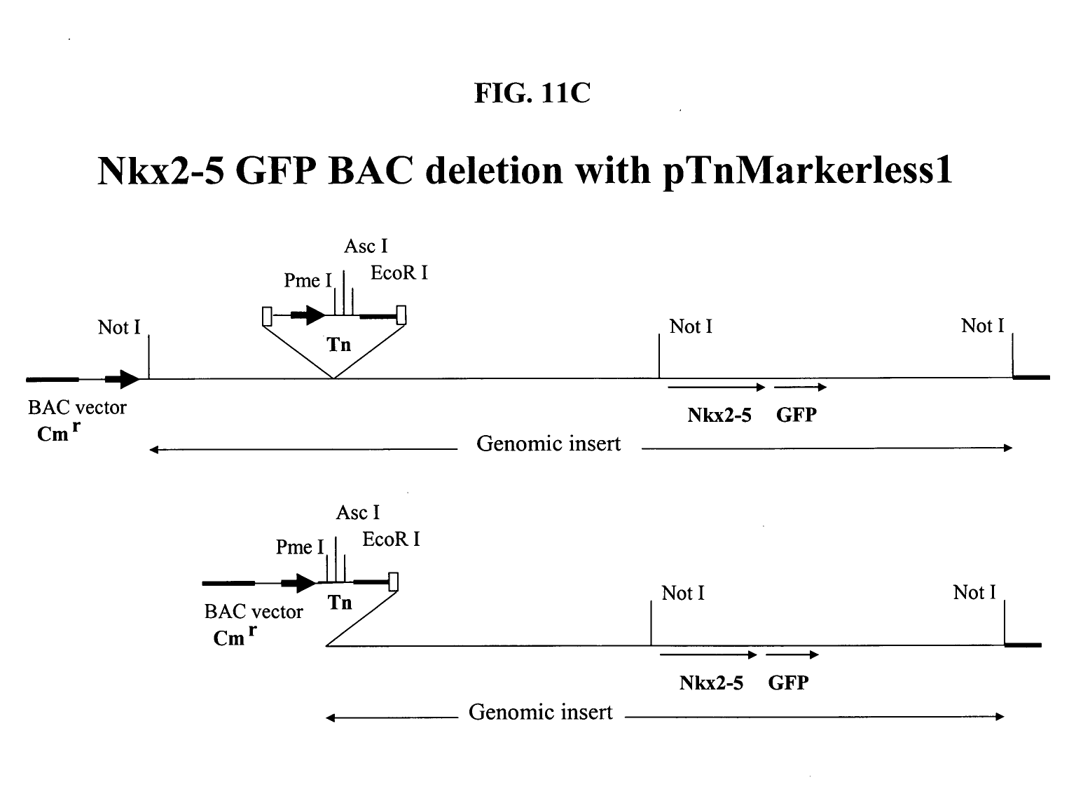

The 1044 bp kanamycin gene cassette was excised from its TA cloning vector pCR2.1 using Asc I, and ligated into pTnMarkerless1 linearized with Asc I. Plasmid clones with added molecular weight of 1044 bp were selected on LB agar plates containing kanamycin, and a panel of these analyzed to establish the orientation of the 1044 bp DNA cassette. It was desirable to have ones with the NotI site away from the loxP sequence in the plasmid, to distinguish vector DNAs from a Cre-mediated deletion clone and starting BAC using a simple NotI digest (see ref 20 for discussion). Tn plasmids with the NotI site distal to the loxP sequence produced a band approximately 1100 bp in length upon digestion with Pme I and NotI enzymes. The new plasmid was named pTnKan (see FIG. 11A).

Construction of pTnMarkerless2 Transposon Plasmid

DNA from transposon plasmid pTn(Minimal)/loxP (9) was digested completely with Pvu II, and the 6.8 kb fragment purified from a 1% agarose gel in 1× TAE buffer using the Qiagen kit. The purified fragment was digested partially with Sca I, and one of the resulting fragments of approximately 6.3 kb was further purified from a 1% agarose gel. This Pvu II-Sca I fragment with blunt ends at both termini, was circularized by ligation with T4 DNA Ligase, and transformed into the NS3516 host strain described earlier (9). The resulting plasmid was named pTnMarkerless2 (FIG. 11A).

Nested Deletions with pTnMarkerless1, pTnMarkerless2 and pTnKan

The new Tn plasmids pTnMarkerless1 and pTnKan were tested initially on several uncharacterized BACs, but the results shown here are with a modified BAC clone containing the mouse Nkx2-5 gene fused to an IRES-GFP cassette (Nkx2-5 GFP BAC) (Chi et al 2003). The Tn plasmid pTnMarkerless1 was used to generate end-deletions also in a PAC clone (Npr3PAC) described earlier (21). The pTnMarkerless2 was used to make deletions in clones from new human PAC libraries constructed in vectors that contained a wild type and a mutant loxP site flanking the insert DNA. Deletions in BACs and PACs were made as in (29).

Clone DNA Isolation and FIGE Analysis

DNA from BAC/PAC deletions were isolated as described earlier and analyzed by FIGE after NotI digestion (20), or with NotI plus Asc I in 1× NE-4 buffer.

DNA Sequencing

Approximately 600 nucleotides of sequence was obtained using primer Seq 1 or Seq 9 located 38 and 240 nucleotides respectively from the transposon end remaining in each BAC or PAC deletion generated with either pTnMarkerless1 or pTnMarkerless2 as described earlier (20). An automated capillary DNA sequencer from ABI (3100 AVANT genetic analyzer) was used with Big Dye Terminator chemistry to obtain sequence reads. A Magnesil (Promega Corporation) based procedure was used to purify primer extended product from template BAC/PAC DNA before applying to capillaries (55). Each sequence read started with fifteen bases from the transposon end.

Results

A series of loxP site-containing Tn10 minitransposons were constructed earlier for use in BACs and PACs (9). While those proved useful in locating markers on a physical map (20, 21), their use in functionally accessing the effect of long range enhancers and silencers in genes has been complicated by the introduction of strong mammalian promoter elements into deletion clones (X Chi and R J Schwartz, unpublished observations). The strong RSV promoter that drives expression of neomycin resistance in pTnBAC/loxP was designed originally to serve as a selectable marker in mammalian cells, and could potentially interfere with a gene regulatory element being mapped in a functional assay. All genes and promoter elements are therefore removed from the transposable part of DNA in the new transposons, and insertions selected by a novel P1 headful packaging procedure illustrated schematically in FIG. 11B.

It became clear during preliminary investigations that using P1 headful packaging to select for transpositions instead of the conventional antibiotic resistance could lead to complications from intra-insert loxP-Cre independent recombinations that also reduce DNA length. Therefore a strategy that verifies BAC vector size in deletion clones after NotI digests was devised to distinguish authentic loxP-Cre mediated deletions from those illegitimate recombination events within the genomic insert. This is shown schematically in FIG. 1C for deletions in the Nkx2-5 GFP BAC clone with the pTnMarkerless 1 transposons. The pTnMarkerless 2 transposon was also designed with the same goal in mind for use in PAC and P1 clones derived from genomic libraries made in vectors with different NotI site characteristics.

As a first step the RSV neomycin gene cassette was removed from pTnBAC/loxP to create pTnMarkerless1 as described in Materials and Methods. Note that NotI digests of authentic LoxP-Cre deletion clone DNA generated with pTnMarkerless1 would not produce the 6.7 kb BAC vector DNA fragment because the recombination event would eliminate the NotI site proximal to the vector loxP sequence (FIG. 11C). Instead, only a single band comprising of both deleted genomic insert and BAC vector DNA would be produced if there are no other NotI sites within the insert DNA.

Nested Deletions with Transposon Plasmid pTnMarkerless1

Tn10 insertion is a relatively infrequent event, occurring at 1 in 10,000 cells for transposon vectors carrying an inducible transposase such as the ones described here (72). Normally transpositions are selected for using resistance to an antibiotic carried by the transposing piece of DNA (73). In the nested deletion procedure, an alternate selection strategy is already in place for insertions of a loxP site: the ability of deletion clones to be packaged into a P1 phage head. If the starting clone or the resulting deletion is larger than 110 kb, the P1 head is unable to package the second loxP site from the P1-BAC/PAC cointegrate, rendering the linear DNA within its head unsalvageable when subsequently infecting a bacterium (see FIG. 1C, and also FIG. 1A of ref 14). Because deletions result from an introduced loxP site, placement of which depends on transposition, one should be able to use P1 headful packaging itself for selecting transpositions without additional resistance markers. This possibility was explored using the TnMarkerless1 transposon.

Progressive deletions from one end of insert DNA were made with pTnMarkerless1 in a BAC clone containing the entire mouse Nkx2-5 gene fused to an IRES-GFP cassette using procedures identical to ones described earlier, with the following changes: First, Nkx2-5 GFP BAC cells were selected after transformation with pTnMarkerless1 on LB plates containing chloramphenicol plus ampicillin. The ampicillin resistance gene, located outside the transposon boundaries, does not insert into its target. Second, pTnMarkerless1 transformed BAC cultures were grown in 12.5 μg/mL chloramphenicol and 60 μg/mL ampicillin prior to IPTG induction. Finally, BAC deletion clones were selected on LB plates containing only chloramphenicol. Deletion libraries obtained with these new transposons were found to have characteristics, such as clone number and distribution of deletion size, similar to those obtained with transposons described previously (14).

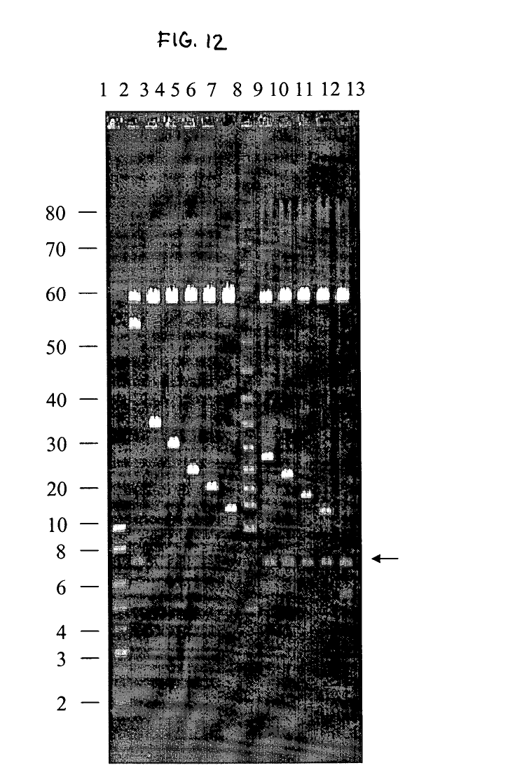

DNA from a panel of deletion clones obtained with pTnMarkerless1 is shown in FIG. 12. The Nkx 2-5 GFP BAC used here contains one NotI site within; and two flanking the insert DNA. Although sixty clones were analyzed from each deletion library, sets of only 5-6 clones covering a range of sizes are presented here. Because the transposon has no NotI site (FIG. 11A), a loxP mediated deletion creates a loss of one NotI site, and the BAC vector DNA is now fused to genomic insert in the deletion (shown schematically in FIG. 11C). Lanes 3-7 in FIG. 12 shows a NotI digest of DNA isolated from such deletion clones. However, the loxP-mediated deletion does introduce a Pme I and an Asc I site from the transposon at approximately the same location as and replacing the NotI site in the starting BAC (FIG. 11C). Therefore a double digest of deletion clone DNA with NotI and either Pme I or Asc I releases the original BAC vector DNA fragment. Because multiple Pme I sites exist in this clone the digestion pattern was complex, although the BAC vector DNA fragment was clearly identified (data not shown). Since there is no Asc I site in the genomic insert, deletion clones digested with both NotI and Asc I in Lanes 9-13 FIG. 12 display the same set of DNA of lanes 3-7, showing the 6.7 kb vector DNA fragment.

pTnMarkerless1 is also Suitable for Generating Nested Deletions in PACs:

A 160 kb PAC containing the human Npr3 gene (21) was transformed with pTnMarkerless1, and cells with both plasmids selected on LB plates containing kanamycin and ampicillin. Deletions were produced as before except that pTnMarkerless1 transformed PAC colonies were grown prior to IPTG induction in kanamycin plus ampicillin, and PAC deletions selected on kanamycin only plates.

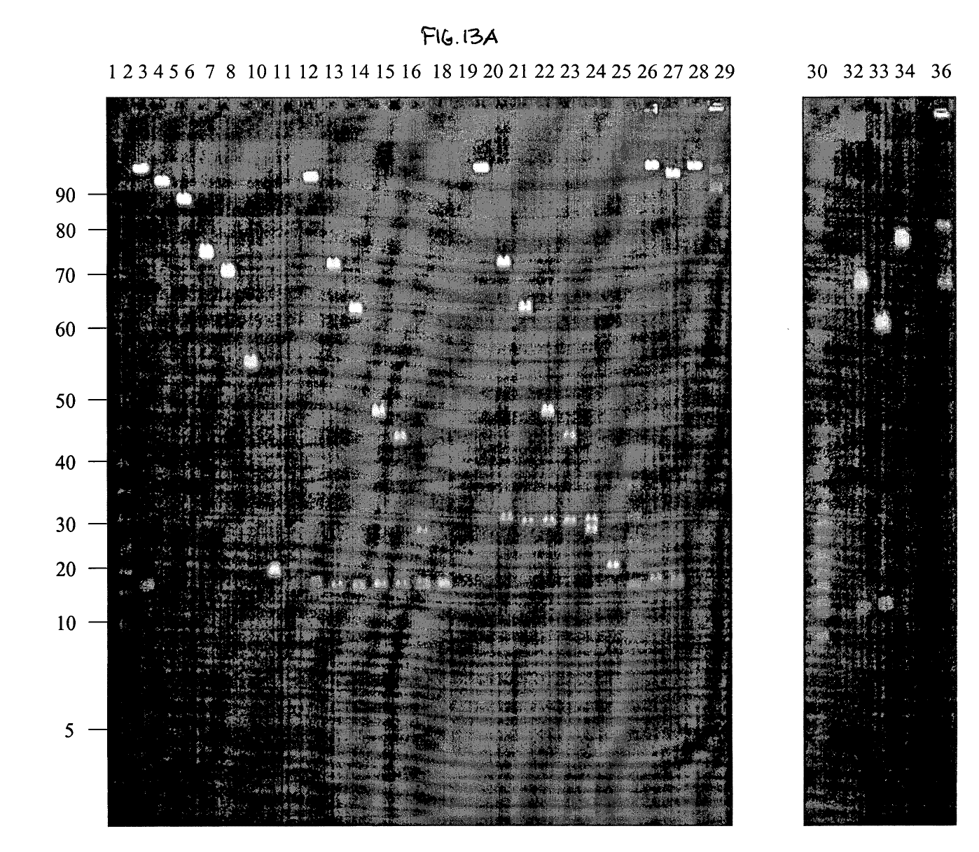

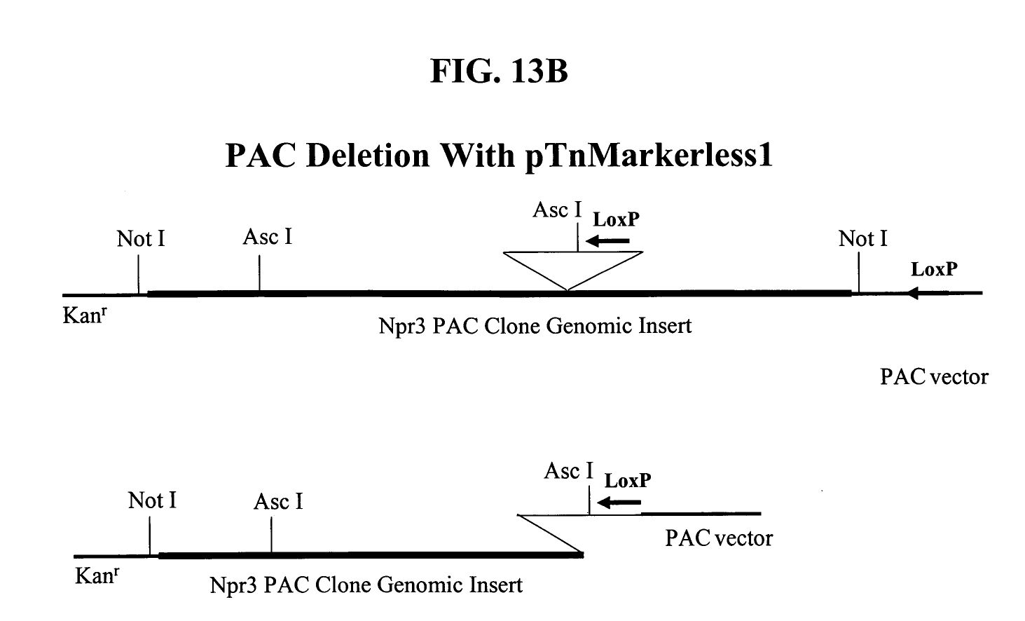

DNA from six deletion clones and starting PAC are displayed in FIG. 13A. A NotI digest is displayed in lanes 2-8. PACs derived from the pCYPAC2 vector contain two NotI sites flanking the insert and allow vector DNA to be excised upon digesting with the enzyme as seen in lane 2. Loss of a NotI site from the loxP-Cre mediated deletion causes this vector band to fuse with the insert as in the case of BAC clones described above. Therefore digestions with NotI alone linearize deletion clone DNA as shown in lanes 3-8. Cre mediated deletion between loxP sites in PACs using pTnMarkerless1 replaces the NotI site proximal to the vector loxP with an Asc I site as in the BACs (FIG. 13B). Therefore digestion of DNA from deletion clones, as well as starting PAC, simultaneously with Asc I and NotI release the PAC vector DNA fragment as shown in lanes 10-16. However, several apparent inconsistencies were noted in the double digests: 1) although a new band appears in deletion clones at the position of vector DNA, the size of inserts in deletion clones are too small for removal of 17 kb vector DNA alone (compare lanes 3-8 with 11-16 respectively), and 2) the starting Npr3PAC now produces a doublet band at the position of vector (compare lanes 2 and 10 or 26 and 27 respectively). The data is consistent with an additional Asc I site in the insert DNA of this clone, located less than 15 kb away from the distal NotI site as indicated in FIG. 13B. Therefore, the DNA fragment located at the position of vector alone in the Asc I- NotI double digests is actually a doublet band in all except the smallest sized deletion clone in lane 16. The combined molecular weights of DNA fragments now match ones in the single digests with NotI. The vector DNA fragment in starting Npr3PAC (lane 10) is slightly larger (by 400 bp) compared to the deletions, and is resolved in these gels. A digest of the same set of DNA samples with Asc I alone shown in lanes 19-25 confirms this hypothesis. The larger DNA fragments in lanes 26 and 28 (NotI and Asc I digests of starting Npr3PAC) appear identical due to poor resolution in this particular FIGE program. Lanes 30 through 36 show the same set of samples as in lanes 26-29 using a different FIGE program.

The analysis of deletion clones generated with pTnMarkerless1 was further substantiated by reintroducing a bacterial kanamycin resistance gene along with a NotI site at the Asc I site in the transposing piece of DNA of pTnMarkerless1. Construction of this new transposon pTnKan (FIG. 11), is described in Materials and Methods. Note that deletion clones generated with pTnKan would produce a vector DNA fragment of 8 kb if mediated by loxP-Cre.

Nested Deletions with pTnKan

Nkx2-5 GFP BAC-containing cells were transformed with pTnKan, and nested deletions generated using the procedures described earlier. Deletion clones resistant to both kanamycin and chloramphenicol were isolated. As a control, nested deletions in Nkx2-5 GFP BAC were also generated in parallel with the previously described transposon plasmid pTnBAC/loxP.

Analysis of NotI digested DNA from pTnKan-generated deletion clones indicate that it can truncate BACs just as efficiently as pTnBAC/loxP. Deletions from illegitimate recombinations within the genomic insert are no longer recovered due to simultaneous selection of transposition with kanamycin resistance. Lanes 7-11 in FIG. 4 display deletions obtained with pTnBAC/loxP, while those from using pTnKan are shown in lanes 3-5 and 12, 13. Lane 2 contains DNA from the starting Nkx2-5 GFP BAC. As noted earlier, the vector DNA fragments from the starting BAC and the deletions generated with pTnBAC/loxP migrate at approximately 6.7 and 8.5 kb respectively (20). The specific increase in size of vector band arises from replacing the BAC vector NotI site proximal to the loxP sequence with the transposon NotI site located approximately 1.7 kb away, and serves as a stringent diagnostic for deletions arising specifically by Cre/loxP recombinations (see FIG. 2 ref 20).

Note that with pTnKan the NotI site that replaces the BAC vector NotI site proximal to the loxP sequence is now only 1.1 kb instead of 1.7 kb away as shown in FIG. 1. Therefore the NotI vector DNA fragment in pTnKan generated deletions is now 8 kb, as shown in lanes 3-5 and 12, 13.

Frequency of Desired vs Undesired Deletions using pTnMarkerless1

In the absence of selection for transposition with an antibiotic resistance marker, all pathways that lead a P1 head to package the two loxP sites in the linear DNA from the phage-BAC/PAC cointegrate is recovered eventually as deletion clones (see FIG. 1A, ref 9). One such pathway is through recombination of sites within the insert DNA, and thus independent of loxP insertions. P1 phage is known to stimulate both plasmid and chromosomal DNA recombination in E. coli by expressing its Ref function (26-28). However it is important to note that both transposition and homologous recombination are each low frequency events that are independent of one another, and therefore a Cre/loxP deletion clone arising through transposition is extremely unlikely to also contain rearrangements originating from intra-insert recombination events (see discussion below).

A stringent procedure to score authentic Cre/loxP mediated deletions was devised: 1) identifying a specific increment in size of the vector band in deletions from both BACs and PACs where the NotI site in the original vector is replaced by one from the transposon (20), and 2) the absence of, and reappearance of vector DNA fragment in digestions of deletion clone DNA with NotI and Asc I respectively. Deletions that originate from intra-insert recombination are thus easily identified with the following characteristics: NotI digests produce vector DNA fragment that is identical in size to the starting BAC or PAC, and insert DNA band is smaller than that of starting clones.

The frequency of intra-insert deletions not involving loxP sites varies widely with the PAC or BAC clone analyzed, and might be a useful measure of the abundance of recombinogenic sites in the genomic DNA in them. Sequence repeats in the insert DNA appear to be a major contributor. Among the several BACs and PACs tested the frequency of intra-insert deletions varied widely, from 3 and 5% in JC-PAC 9 and JC-PAC13 respectively, 15% and 20% in Npr3PAC and Thox 18 BAC clone, to about 40% for Nkx2-5 GFP BAC. In one uncharacterized BAC clone it reached a high of 70% (data not shown). This is probably not surprising since studies have identified 200-300 bp sequences repeated numerous times in either orientation at several locations in a single large clone (21). Similar repeats also exist in the Nkx2-5 GFP BAC.

Nested Deletions with pTnMarkerless2

A second transposon, pTnMarkerless2, with slightly different restriction site characteristics (FIG. 11) was constructed to facilitate identification of end-deletions generated in clones where the vector does not contain NotI sites on either end of insert DNA. The shuttle vector pJCPAC-Mam1 (29), the P1 vector (30, 31), and the pair pJCPAC-Mam2 A & B, with wild type and mutant loxP sites flanking the insert DNA, fall in this category.

FIG. 10 shows two panels of nested-deletion clone DNA digested with NotI enzyme and analyzed by FIGE. The deletions were obtained using pTnMarkerless2 on either a 115 kb clone in pJCPAC-Mam2A vector (lanes 3-15), or a 140 kb clone in the pJCPAC-Mam2B vector (lanes 20-30). The two vectors contain a wild type loxP site and one of two different mutant loxP sites flanking the insert DNA in the clones. An authentic wt loxP-Cre deletion with pTnMarkerless2 generates a vector DNA fragment of 14.5 kb due to the genomic insert DNA acquiring a NotI site from TnMarkerless2 as a consequence of the LoxP-Cre recombination (FIG. 11). Lanes 2 and 19 show the NotI digests of the starting clones from the two vector systems respectively, and they contain no vector DNA fragment. The few clones arising through intra-insert recombination independent of Cre-loxP (3 and 5% respectively) did not produce this vector DNA fragment either. All deletions with pTnMarkerless2 in the two PAC clones were exclusively from the wild type loxP end: all clones produced good sequence reads with the transposon-end primer and aligned with sequences in GeneBank exclusively to that strand consistent with deletions from the wild type loxP end upon BLAST analysis. In contrast the intra-insert Cre-loxP independent deletions produced no sequence reads with the transposon-end primer.

Discussion

Mice containing entire BACs as transgenes are being increasingly used for studying regulation of tissue specific genes (58, 59, 62, 71). Much of this regulation is at the transcriptional level, and appears mediated by sequence elements far upstream of the gene. Introducing BACs or PACs truncated from one or both ends of the insert DNA into animals should facilitate mapping such long-range regulatory sequences in vivo because they are less likely to be influenced by site of chromosomal integration. Getting modified BAC DNA into mice embryos to generate transgenic animals do not require a mammalian cell selectable marker in the clone DNA. Therefore the strong promoter elements driving expression of selectable markers inserted during retrofitting are no longer necessary. In fact their presence is likely to interfere with one's ability to functionally identify transcription enhancing sequences and mask potential silencers that are endogenous to the gene in the clone (Chi et al unpublished observations). Several new transposon plasmids were therefore constructed to overcome such potential hurdles in localizing complex long-range gene regulatory sequences in BACs and PACs. One of these, pTnMarkerless1, has been successfully used to identify three new enhancer elements 27 kb upstream of the Nkx2-5 gene in transgenic mice (X. Chi et al submitted).

The markerless transposons described here, pTnMarkerless1 and 2, use a novel strategy to select for insertions: instead of screening for a growth advantage conferred by the transposed piece of DNA upon the target, such as expression of antibiotic resistance, we have relied on the inserted loxP sequence to reduce BAC-DNA length via loxP-Cre recombination to enable a P1 phage to package both loxP sites of the linear BAC DNA within its head (illustrated schematically in FIG. 11C). Because the strategy does not use selection for antibiotic resistance, the same transposons could be used for both BACs and PACs.

Circumventing positive selection for a marker on the transposing piece of DNA results in isolating clones of reduced size from events both loxP/Cre mediated as well as those unrelated to loxP transposition: BAC deletions arising from intra-insert DNA recombination also get rescued by P1 headful packaging if their size is less than 110 kb. Such deletions, mediated presumably by homologous recombination of sequence repeats within the genomic insert, occur more frequently upon P1 infection than during the course of normal propagation of a BAC clone due to expression of the phage-encoded Ref gene (75). The findings might also help explain why small numbers of colonies are occasionally recovered during P1 transduction of larger than 110 kb BACs without transposon insertions (PKC unpublished observations). The Ref-induced enhancement in homologous recombination is between 3-6 fold and is dependent on pre-existing E. coli recombination functions (75), many of which are rendered deficient in hosts such as DH10B for stable propagation of BAC/PAC plasmids. It is important to note however that both induced-transposition and Ref-enhanced recombination are very low frequency events, of the order of 10⁻⁴ and 10⁻⁷, respectively (80). The question of relevance here is whether an intra-insert Ref-induced recombination might rearrange and complicate an otherwise good loxP-mediated deletion. Such a scenario is expected to be extremely unlikely, and is borne out by observations reported earlier about rearranged clones being substantially less than 1% of isolates (20).

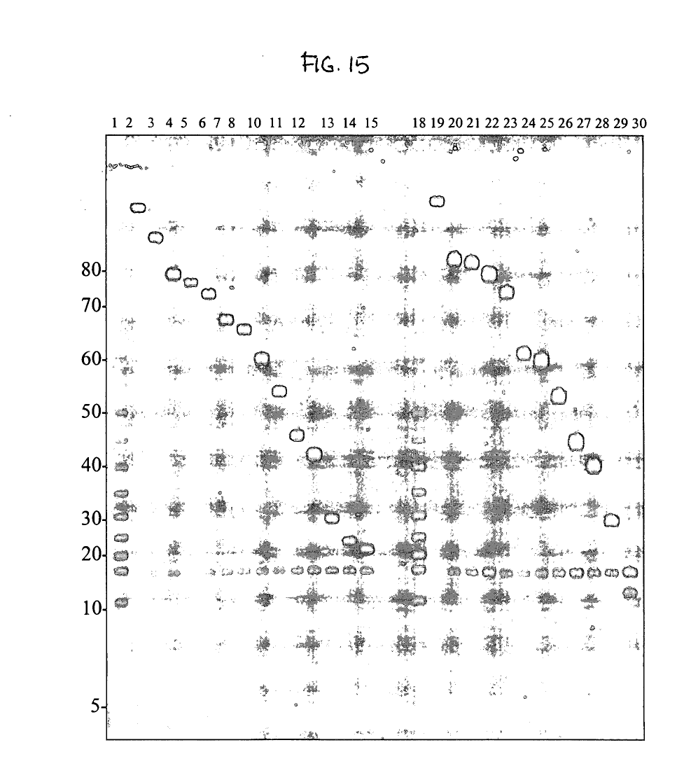

A key to the usefulness of markerless and other loxP transposons is the stringent criteria used to identify authentic Cre-loxP deletions: a BAC vector DNA fragment of specific size with NotI in deletion clone DNA obtained with pTnKan or pTnBAC/loxP (FIG. 4) (see also ref 20), replacement of a NotI site with Asc I and Pme I sites using pTnMarkerless1 with standard BACs and PACs (FIGS. 12 & 13), or introducing a new NotI site adjacent to the wt loxP in the JC-PAC bi-directional shuttle vector clones with pTnMarkerless2 (FIG. 15).

Residual antibiotic resistance genes are often an unavoidable consequence of functionalizing BACs and PACs, and given the scarcity of suitable antibiotic resistance marker genes, the markerless transposons described here are likely to facilitate making nested deletions in these so as to characterize such modified genomic clones by end-sequencing parts of it. The P1 headful packaging criteria for selecting transposition of loxP sites should in principle allow earlier transposons such as pTn(Minimal)/loxP or pTnPGKpuro/loxP (14), designed originally for PACs, to generate nested end-deletions also in BACs. In practice however, the Tn plasmid is recovered at a much greater frequency compared to deleted BAC clones because of its higher copy number in cells: both plasmids contain the same selectable marker (chloramphenicol resistance) and the loxP site for forming cointegrates with P1 vir phage and thus packaged by it (see ref 9 for detailed discussion of complications earlier with pTnRSVneo/loxP). The TnMarkerless transposons do not have this complication, as we select only for the marker in the BAC (or PAC) when screening for deletion clones, and the Tn plasmid is selected against. However, it needs to be emphasized that selecting transpositions using P1 headful packaging alone is feasible only for BACs and PACs that are larger than 110 kb. Fortunately, most clones in current BAC/PAC libraries are much larger, with an average insert size of 160-230 kb (8).

FIG. 11A. Schematic Representation of Eucaryotic-Promoterless Transposons

Reference: is made to FIGS. 11A-11C, wherein the thick arrows represent the wild type loxP site. The small open vertical rectangles represent the transposon boundaries, while the elongated open rectangles designate genes. Tn plasmids pTnBAC/loxP and pTn(minimal)/loxP have been described (ref 14). FIG. 11B shows a schematic representation explaining how the markerless transposon strategy works: Cre protein mediated recombination allows a cointegrate to be formed between the phage P1 DNA and the BAC DNA. The “pac site” in the cointegrate is recognized and cleaved by the P1 packaging machinery, and the end is stuffed into P1 empty heads. Packaging continues in the order B, D, E, F, till the P1 head is full at around 110 kb of DNA. The remaining DNA outside the head is then chopped off in a sequence non-specific manner by what is called “headful-cleavage”. The recoverability of the DNA within the P1 head depends on whether the second loxP site (thick arrow head) is also included in the head. Upon subsequent infection, the linear DNA in the phage enters the cell, and is circularized by newly expressed Cre protein using the two loxP sites. If the second loxP site is not there, the linear DNA is destroyed. The packaging of both loxP sites within a P1 head thus depends on shortening the length of DNA D, E, F, to less than 110 kb. One of the ways this can happen is by the insertion of a loxP sequence in the correct orientation through transposition. If the starting BAC or PAC is larger than 110 kb, the recovery of a deletion clone therefore serves as a selection for transposition of a loxP site. (C) Schematic diagram showing the loxP-transposon induced deletion in Nkx2-5 GFP BAC with pTnMarkerless1. The diagnostic restriction site patterns are illustrated.

FIG. 12. FIGE Analysis of Nkx2-5 GFP BAC Deletions Generated with pTnMarkerless1

Referring now to FIG. 12, the DNA from the same set of five clones from a deletion series generated with pTnMarkerless1 are shown after digestion with NotI enzyme in lanes 3-7, and after NotI plus Asc I digestion in lanes 9-13. Lanes 1 and 8 show a 1 kb and a 5 kb ladder, respectively. Lane 2 contains starting NkxBAC digested with NotI enzyme. The arrow locates vector DNA fragment in starting BAC and deletion clones.

FIG. 13. (A) FIGE Analysis of Npr3PAC Deletions Generated with pTnMarkerless1

Referring now to FIG. 11A ns FIG. 11B, there is shown the DNA from a set of six deletion clones that was digested with NotI enzyme (lanes 3-8), NotI plus Asc I enzymes (lanes 11-16) and Asc I enzyme alone (lanes 19-24). The starting Npr3PAC clone DNA digested with NotI is shown in lanes 2, 26 and 32; with NotI plus Asc I enzymes in lanes 10, 27 and 33; and with Asc I alone in lanes 18, 28 and 34. Lanes 29 and 36 show the starting Npr3PAC DNA not digested with any enzyme. A 5 kb ladder was applied to lanes 1, 25 and 30. Lanes 1-29 was run on FIGE program 6, while 30-36 was run on program 8. (B) A schematic representation of the starting Npr3PAC clone and its deletion obtained using pTnMarkerless1.

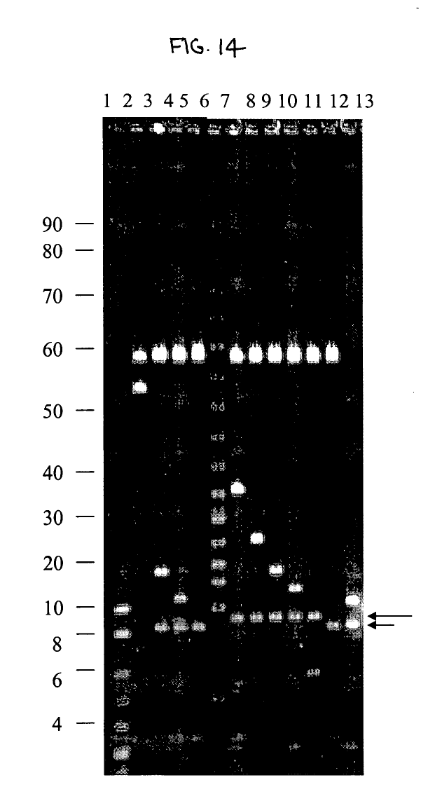

FIG. 14. FIGE Analysis of NotI Digested DNA from Nkx2-5 GFP BAC Nested Deletions

Referring now to FIG. 14, the DNA from five clones of a nested deletion series generated with either pTnBAC/loxP (lanes 7-11) or pTnKan (lanes 3-5 & 12-13) were digested with NotI enzyme and analyzed by FIGE. Lanes 1 and 6 contain 1 kb and 5 kb ladders as molecular weight standards respectively, and the full length starting NkxBAC clone DNA is shown in lane 2. The large and small arrows on the right indicate positions of the vector DNA fragments seen in deletions made with pTnBAC/loxP deletions (8.5 kb) and pTnKan (8 kb), respectively.

FIG. 15. FIGE Analysis of NotI Digested DNA Isolated from Deletion Clones Generated with pTnMarkerless2 on JCPAC 9 (lanes 3-15) and JCPAC 13 (lanes 20-30)

Now referring to FIG. 15, there is shown the lanes 2 and 19 containing the NotI digested starting clones JCPAC 9 and JCPAC 13 respectively. Lanes 1 and 18, also shown in FIG. 15 contain 5 kb ladders as standards.

TABLE I

| | | | | after | |
|---|---|---|---|---|---|
| | | | | transduction | |
| | | | | with phage P1 | |
| Clone | Size (kb) | Marker | LoxP site | kan | cam |
| #17 | 12 | camR | wt loxP only | N/A | +++++ |
| #21 | 8 | kanR | loxP511 only | + | N/A |
| #39 | 40 | camR | wt plus loxP511 | N/A | +++++ |
| #45 | 60 | camR | wt plus loxP511 | N/A | +++++ |

| Transformed | | |
|---|---|---|
| clones | | |
| #39/21 | +++++ | +++++ |
| #45/21 | +++++ | +++++ |

˜5 colonies/plate: +
˜1000 colonies/plate: +++++
wt loxP ATAACTTCGTATA GCATACAT TATACGAAGTTAT
loxP511 ATAACTTCGTATA GTATACAT TATACGAAGTTAT

TABLE 2

| | | | | Transduction | Transformed | after P1 transduction | | |
|---|---|---|---|---|---|---|---|---|
| Clone | Size (kb) | Marker | LoxP site | with phage P1 | clones | kan | cam | tet |
| #17 | 12 | camR | wt loxP only | ++++ | | | | |
| #18 | 10 | tetR | loxP*-1 only | 0 | | | | |
| #19 | 10 | tetR | loxP*-2 only | 0 | | | | |
| #2 | 85 | kanR | wt loxP only | ++++ | #2/17 | ++++ | ++++ | N/A |
| | | | | | #2/18 | ++++ | N/A | 0 |
| | | | | | #2/19 | ++++ | N/A | 0 |
| #5 | 120 | kanR | wt loxP only | 0 | #5/17 | 0 | ++++ | N/A |
| | | | | | #5/18 | 0 | N/A | 0 |
| | | | | | #5/19 | 0 | N/A | 0 |
| #8 | 60 | kanR | wt plus loxP*-2 | ++++ | #8/17 | ++++ | ++++ | N/A |
| | | | | | #8/18 | ++++ | N/A | 0 |
| | | | | | #8/19 | ++++ | N/A | ++++ |
| #9 | 120 | kanR | wt plus loxP*-2 | 0 | #9/17 | 0 | ++++ | N/A |
| | | | | | #9/18 | 0 | N/A | 0 |
| | | | | | #9/19 | 0 | N/A | ++ |
| #12 | 60 | kanR | wt plus loxP*-1 | ++++ | #12/17 | ++++ | ++++ | N/A |
| | | | | | #12/18 | ++++ | N/A | ++++ |
| | | | | | #12/19 | ++++ | N/A | 0 |
| #13 | 140 | kanR | wt plus loxP*-1 | 0 | #13/17 | 0 | ++++ | N/A |
| | | | | | #13/18 | 0 | N/A | ++ |
| | | | | | #13/19 | 0 | N/A | 0 |

0-3 colonies/plate: 0
50-100 colonies/plate: ++
500-1000 colonies/plate: ++++

What is claimed is:

1. A method of truncating both ends of a DNA insert flanked by a wild type loxP site and a loxP511 site in a vector using loxP transposon plasmids comprising:
a. Constructing a plurality of plasmids of identical sequence each containing a first transposon carrying a loxP511 site;
b. Transforming said plurality of first transposon containing plasmids into cells, each containing a DNA insert in said vector carrying a first antibiotic resistance gene;
c. Inducing each of said plurality of first transposons to insert into said DNA inserts in said cells;
d. Infecting said cells containing said plurality of first transposons inserted into said DNA inserts with Phage P1, and wherein Cre protein is expressed by Phage P1 during said infection;
e. Truncating first end portions of each of said DNA inserts, wherein each of said DNA inserts contains one of said inserted first transposons, and thereby creating a plurality of first end truncated DNA inserts with new first ends;
f. Packaging said first end truncated DNA inserts into a P1 Phage head;
g. Using said P1 Phage head to infect a first colony of fresh NS3516 cells to amplify said first end truncated DNA inserts by selecting on LB agar plates containing a first antibiotic whose resistance gene occurs in said vector, and thereby amplifying said plurality of first end truncated DNA inserts in a plurality of NS3516 cell colonies generated from said first colony of fresh NS3516 cells;
h. Isolating said amplified plurality of different first end truncated DNA inserts in said vector to obtain a plurality of colonies, each colony with the same end truncation, from said first colony of fresh NS3516 cells;
i. Using a first synthetic DNA primer to sequence said amplified plurality of new first end portions of said amplified plurality of different first end truncated DNA inserts;
j. Constructing a plurality of plasmids of identical sequence each containing a second transposon carrying a wild type loxP site and a second antibiotic resistance gene different from said first antibiotic resistance gene occurring in said vector;
k. Transforming said plurality of second transposon containing plasmids into cells containing said amplified plurality of first end truncated DNA inserts;
l. Inducing said plurality of second transposons to insert into said amplified plurality of first end truncated DNA inserts;
m. Infecting said cells containing said plurality of second transposons inserted into said plurality of amplified first end truncated DNA inserts with Phage P1, and wherein Cre protein is expressed by Phage P1 during said infection;
n. Truncating an opposite end portion of each of said plurality of amplified first end truncated DNA inserts, wherein each of said amplified first end truncated DNA inserts contains one of said inserted second transposons and creating a plurality of doubly end truncated DNA inserts with a new opposite end;
o. Packaging said plurality of doubly end truncated DNA inserts in said vector into said P1 Phage head;
p. Using said P1 Phage head to infect a second colony of fresh NS3516 cells to amplify said plurality of doubly end truncated DNA inserts by selecting on LB agar plates containing said first antibiotic and a second antibiotic whose resistance gene exists in said second transposon, and thereby amplifying a plurality of doubly end truncated DNA inserts in a plurality of NS3516 cell colonies generated from said second colony of fresh NS3516 cells;
q. Isolating said amplified plurality of different doubly end truncated DNA inserts in said vector to obtain a plurality of colonies, each colony with the same opposite end truncation from said second colony of fresh NS3516 cells; and
r. Using a second synthetic DNA primer to sequence said amplified plurality of new opposite end portions of said different doubly end truncated DNA inserts.

2. The method of claim 1 wherein said first transposon is transposon plasmid pTnloxP511 (B) markerless 1.

3. The method of claim 1 wherein said first transposon is transposon plasmid pTnloxP511 (B) markerless 2.

4. The method of claim 2 wherein said second transposon is pTn(RSVneo 2)/loxP.

5. The method of claim 3 wherein said second transposon is pTn(RSVneo 2)/loxP.

6. The method of claim 2 wherein said second transposon is pTn(RSVneo 1)/loxP.

7. The method of claim 3 wherein said second transposon is pTn(RSVneo 1)/loxP.

8. The method of claim 1 wherein said first transposon is transposon plasmid pTnloxP511 (A) markerless 2.

9. The method of claim 8 wherein the second transposon is pTn(RSVneo 2)/loxP.

10. The method of claim 8 wherein the second transposon is pTn(RSVneo 1)/loxP.

11. The method of claim 1 wherein the first transposon is transposon plasmid pTnloxP511 (B) markerless 1 and wherein said first antibiotic is chloramphenicol and said second antibiotic is kanamycin.

12. The method of claim 1 wherein the vector is a BAC clone selected from the group consisting of pBACe3.6, pTARBAC 1 & 2, and pTARBAC 2.1.

13. The method of claim 1 wherein the vector is a BAC clone selected from the group consisting of RP11-219A15, RP11-158M20, RP11-434D2, RP23-209O22, RP23-92L23, RP23-366M16, RP23-101N20, RP23-124B2, RP23-444K15, and RP23-130D16.

14. The method of claim 2 wherein the first synthetic DNA primer is DNA oligonucleotide Sequence 1.

15. The method of claim 3 wherein the first synthetic DNA primer is DNA oligonucleotide Sequence 1.

16. The method of claim 4 wherein the second synthetic DNA primer is a DNA oligonucleotide chosen from the group Sequence 4 and Sequence 6.

17. The method of claim 6 wherein the second synthetic DNA primer is a DNA oligonucleotide chosen from the group Sequence 4 and Sequence 6.

18. A method of truncating both ends of a DNA insert flanked by two different loxP sequences using transposons carrying corresponding loxP sequences pertaining to said two ends.

19. A method of truncating both ends of a DNA insert flanked by a wild type loxP site and a loxP511 site in a vector using loxP transposon plasmids comprising:
a. Constructing a plurality of plasmids of identical sequence each containing a first transposon carrying a wild type loxP site;
b. Transforming said plurality of first transposon containing plasmids into cells, wherein each containing a DNA insert in said vector carrying a first antibiotic resistance gene;
c. Inducing each of said plurality of first transposons to insert into said DNA inserts in said cells;
d. Infecting said cells containing said plurality of first transposons inserted into said DNA inserts with Phage P1, and wherein Cre protein is expressed by Phage P1 during said infection;
e. Truncating first end portions of each of said DNA inserts, wherein each of said DNA inserts contains one of said inserted first transposons, and thereby creating a plurality of first end truncated DNA inserts with new first ends;
f. Packaging said first end truncated DNA inserts into a P1 Phage head;
g. Using said P1 Phage head to infect a first colony of fresh NS3516 cells to amplify said first end truncated DNA inserts by selecting on LB agar plates containing a first antibiotic whose resistance gene occurs in said vector, and thereby amplifying said plurality of first end truncated DNA inserts in a plurality of NS3516 cell colonies generated from said first colony of fresh NS3516 cells;
h. Isolating said amplified plurality of different first end truncated DNA inserts in said vector to obtain a plurality of colonies, each colony with the same end truncation, from said first colony of fresh NS3516 cells;
i. Using a first synthetic DNA primer to sequence said amplified plurality of new first end portions of said amplified plurality of different first end truncated DNA inserts;
j. Constructing a plurality of plasmids of identical sequence each containing a second transposon carrying a loxP511 site and a second antibiotic resistance gene different from said first antibiotic resistance gene occurring in said vector;
k. Transforming said plurality of second transposon containing plasmids into cells containing said amplified plurality of first end truncated DNA inserts;
l. Inducing said plurality of second transposons to insert into said amplified plurality of first end truncated DNA inserts;
m. Infecting said cells containing said plurality of second transposons inserted into said plurality of amplified first end truncated DNA inserts with Phage P1, and wherein Cre protein is expressed by Phage P1 during said infection;
n. Truncating an opposite end portion of each of said plurality of amplified first end truncated DNA inserts, wherein each of said amplified first end truncated DNA inserts contains one of said inserted second transposons and creating a plurality of doubly end truncated DNA inserts with a new opposite end;
o. Packaging said plurality of doubly end truncated DNA inserts in said vector into said P1 Phage head;
p. Using said P1 Phage head to infect a second colony of fresh NS3516 cells to amplify said plurality of doubly end truncated DNA inserts by selecting on LB agar plates containing said first antibiotic and a second antibiotic whose resistance gene exists in said second transposon, and thereby amplifying a plurality of doubly end truncated DNA inserts in a plurality of NS3516 cell colonies generated from said second colony of fresh NS3516 cells;
q. Isolating said amplified plurality of different doubly end truncated DNA inserts in said vector to obtain a plurality of colonies, each colony with the same opposite end truncation from said second colony of fresh NS3516 cells; and
r; Using a second synthetic DNA primer to sequence said amplified plurality of new opposite end portions of said different doubly end truncated DNA inserts.

20. The method of claim 19 wherein said first transposon is transposon plasmid pTn markerless 2.

21. The method of claim 20 wherein said second transposon is a transposon plasmid chosen from the group pTnloxP511 (B)RSVneo 2.

22. The method of claim 19 wherein the first synthetic DNA primer is a DNA oligonucleotide chosen from the group Sequence 4 and Sequence 6.

23. A method of selecting transpositions using phage P1 headful packaging instead of using an antibiotic resistance gene within a transposon comprising:
a. Constructing a plurality of plasmids of identical sequence each containing a first transposon carrying either a wild type loxP or a mutant loxP site;
b. Transforming said plurality of first transposon containing plasmids into cells, each containing a DNA insert in said vector carrying a first antibiotic resistance gene;
c. Inducing each of said plurality of first transposons to insert into said DNA inserts in said cells;
d. Infecting said cells containing said plurality of first transposons inserted into said DNA inserts with Phage P1, and wherein Cre protein is expressed by Phage P1 during said infection;
e. Truncating first end portions of each of said DNA inserts, wherein each of said DNA inserts contains one of said inserted first transposons, and thereby creating a plurality of first end truncated DNA inserts with new first ends;
f. Packaging said first end truncated DNA inserts into a P1 Phage head;
g. Using said P1 Phage head to infect a first colony of fresh NS3516 cells to amplify said first end truncated DNA inserts by selecting on LB agar plates containing a first antibiotic whose resistance gene occurs in said vector, and thereby amplifying said plurality of first end truncated DNA inserts in a plurality of NS3516 cell colonies generated from said first colony of fresh NS3516 cells;
h. Isolating said amplified plurality of different first end truncated DNA inserts in said vector to obtain a plurality of colonies, each colony with the same end truncation, from said first colony of fresh NS3516 cells; and
i. Using a first synthetic DNA primer to sequence said amplified plurality of new first end portions of said amplified plurality of different first end truncated DNA inserts.


SEQUENCE LISTING (21 sequences)

SEQ ID NO: 1 - DNA, 50 bp, E. coli phage P1 mutant sequence
  ref <301>: AUTHORS: Chatterjee, PK et al.
  ref <302>: TITLE: Mutually Exclusive ...
  ref <303>: JOURNAL: Nuc. Acids Res.
  ref <304>: VOLUME: 32
  ref <306>: PAGES: 5668-5676
  ref <307>: DATE: 2004
ggccgcataacttcgtataatgtgtactatacgaagttatgtttaaacgc

SEQ ID NO: 2 - DNA, 50 bp, E. coli phage P1 mutant sequence
  ref <301>: AUTHORS: Chatterjee, PK, et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
ggccgcataacttcgtataaagtatcctatacgaagttatgtttaaacgc

SEQ ID NO: 3 - DNA, 20 bp, E. coli transposon Tn10 mutant sequence
  ref <301>: AUTHORS: Chatterjee, PK, et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
cttccatgtcggcagaatgc

SEQ ID NO: 4 - DNA, 22 bp, E. coli transposon Tn10 mutant sequence
  ref <301>: AUTHORS: Chatterjee, PK et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
gttcatcatgccgtctgtgatg

SEQ ID NO: 5 - DNA, 21 bp, E. coli transposon Tn10 mutant sequence
  ref <301>: AUTHORS: Chatterjee, PK, et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
cgctggcgattcaggttcatc

SEQ ID NO: 6 - DNA, 21 bp, E. coli transposon Tn10 mutant sequence
  ref <301>: AUTHORS: Chatterjee, PK, et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
caaggcgacaaggtgctgatg

SEQ ID NO: 7 - DNA, 31 bp, E. coli transposon Tn10 mutant sequence
  ref <301>: AUTHORS: Chatterjee, PK, et al.
  ref <302>: TITLE: Selecting Transpositions...
  ref <303>: JOURNAL: Anal. Biochem.
  ref <304>: VOLUME: 335
  ref <306>: PAGES: 305-315
  ref <307>: DATE: 2004
ggcgcgccgcggccgcgtgatctgatccttc

SEQ ID NO: 8 - DNA, 26 bp, E. coli transposon Tn10 mutant sequence
  ref <301>: AUTHORS: Chatterjee, PK, et al.
  ref <302>: TITLE: Selecting Transpositions...
  ref <303>: JOURNAL: Anal. Biochem.
  ref <304>: VOLUME: 335
  ref <306>: PAGES: 305-315
  ref <307>: DATE: 2004
ggcgcgccgtcaagtcagcgtaatgc

SEQ ID NO: 9 - DNA, 52 bp, E. coli phage P1 mutant sequence
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
ggccgctaataacttcgtatagtatacattatacgaagttattgtttaaacc

SEQ ID NO: 10 - DNA, 54 bp, E. coli transposon Tn10 mutant sequence
  ref <301>: AUTHORS: Shakes, LA, et al.
  ref <302>: TITLE: Minimal Cross-recombinatin...
  ref <303>: JOURNAL: Nuc. Acids Res.
  ref <304>: VOLUME: 33
  ref <306>: PAGES: e118
  ref <307>: DATE: 2005
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
cgcgcctagtaaaacgacggccagtagtcgtgactgggaaaacctgtttaaacc

SEQ ID NO: 11 - DNA, 54 bp, E. coli transposon Tn10 mutant sequence
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
cgcgggtttaaacaggttttcccagtcacgactactggccgtcgttttactagg

SEQ ID NO: 12 - DNA, 28 bp, E. coli transposon Tn10 mutant sequence
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
gcagtgtgaccgtgtgcttctcaaatgc

SEQ ID NO: 13 - DNA, 18 bp, E. coli transposon Tn10 mutant sequence
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
gatcggcgcgccatgatc

SEQ ID NO: 14 - DNA, 17 bp, E. coli transposon Tn10 mutant sequence
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
gccagtagtcgtgactg

SEQ ID NO: 15 - DNA, 21 bp, Expression vector RSV.5 (neo)
  ref <301>: AUTHORS: Long, EO, Rosen-Bronson, S. et al.
  ref <302>: TITLE: Efficient cDNA Expression...
  ref <303>: JOURNAL: Hum. Immunol.
  ref <304>: VOLUME: 4
  ref <306>: PAGES: 229-35
  ref <307>: DATE: 1991
gttttcccttgtccagatagc

SEQ ID NO: 16 - DNA, 19 bp, Expression vector RSV.5 (neo)
  ref <301>: AUTHORS: Long, EO, Rosen-Bronson, S, et al.
  ref <302>: TITLE: Efficient cDNA Expression...
  ref <303>: JOURNAL: Hum. Immunol.
  ref <304>: VOLUME: 4
  ref <306>: PAGES: 229-35
  ref <307>: DATE: 1991
ctgagtgcttgcggcagcg

SEQ ID NO: 17 - DNA, 18 bp, Expression vector RSV.5 (neo)
  ref <301>: AUTHORS: Long, EO, Rosen-Bronson, S, et al.
  ref <302>: TITLE: Efficient cDNA Expression...
  ref <303>: JOURNAL: Hum. Immunol.
  ref <304>: VOLUME: 4
  ref <306>: PAGES: 229-35
  ref <307>: DATE: 1991
gaatcgttttccgggacg

SEQ ID NO: 18 - DNA, 24 bp, Expression vector RSV.5 (neo)
  ref <301>: AUTHORS: Long, EO, Rosen-Bronson, S. et al.
  ref <302>: TITLE: Efficient cDNA Expression...
  ref <303>: JOURNAL: Hum. Immunol.
  ref <304>: VOLUME: 4
  ref <306>: PAGES: 229-35
  ref <307>: DATE: 1991
gatctcatgctggagttcttcgcc

SEQ ID NO: 19 - DNA, 52 bp, E. coli phage P1 mutant sequence
  misc_feature (1)..(52): sequence is complimentary to SEQ ID NO:9
  misc_feature (1)..(52): sequence is complimentary to most of SEQ ID NO:9
  ref <301>: AUTHORS: Shakes, LA, et al.
  ref <302>: TITLE: Minimal Cross-recombination...
  ref <303>: JOURNAL: Nuc. Acids Res.
  ref <304>: VOLUME: 33
  ref <306>: PAGES: e118
  ref <307>: DATE: 2005
ggccggtttaaacaataacttcgtataatgtatactatacgaagttattagc

SEQ ID NO: 20 - DNA, 50 bp, E. coli phage P1 mutant sequence
  misc_feature (1)..(50): sequence is complimentary to most of SEQ ID NO:1
  ref <301>: AUTHORS: Chatterjee, PK, et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
ggccgcgtttaaacataacttcgtatagtacacattatacgaagttatgc

SEQ ID NO: 21 - DNA, 50 bp, E. coli phage P1 mutant sequence
  misc_feature (1)..(50): sequence is complimentary to most of SEQ ID NO:2
  ref <301>: AUTHORS: Chatterjee, PK, et al.
  ref <302>: TITLE: Mutually Exclusive...
  ref <303>: JOURNAL: Nuc. Acids Res.
  ref <304>: VOLUME: 32
  ref <306>: PAGES: 5668-5676
  ref <307>: DATE: 2004
ggccgcgtttaaacataacttcgtataggatactttatacgaagttatgc